SEQUENCE LISTING (29 sequences)

SEQ ID NO: 1 - protein, 241 aa, Canis lupus
MSMLFYTLITALLIGIRAEPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 2 - protein, 241 aa, Felis catus
MSMLSYTLITALLIGIQAEPHPESNVPAGHAIPQAHWTKLQHSLDTALRRARSTPAGAIAARVAGQTRNITVDPKLFKKRRLRSPRVLFSTHPPPVAADTQGLDLEAGGAASFNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 3 - protein, 241 aa, Equus caballus
MSMLFYTLITALLIGTQAEPHTESNVPAGHAIPQAHWTKLQHSLDTALRRARSAPARAIAARVAGQTRNITVDPKLFKKRRLRSPRVLFSTQPPPVAADTQDLDFEAGGAASFNRTHRSKRSSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKTGRKA

SEQ ID NO: 4 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 5 - protein, 121 aa, Canis lupus
MSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 6 - protein, 121 aa, Felis catus; identical to SEQ ID NO: 5

SEQ ID NO: 7 - protein, 121 aa, Equus caballus
MSSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKTGRKA

SEQ ID NO: 8 - protein, 121 aa, Homo sapiens
MSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 9 - protein, 11 aa, synthetic construct
RPKPQQFFGLM

SEQ ID NO: 10 - protein, 37 aa, Canis lupus
SCNTATCVTHRLAGLLSRSGGVVKNNFVPTNVGSEAF

SEQ ID NO: 11 - protein, 38 aa, Felis catus
KRCSNLSTCVLGAYSKDLNNFHTFSGIGFGAETPGKKR

SEQ ID NO: 12 - protein, 37 aa, Equus caballus
SCNTASCLTHRLAGLLSSAGSMANSNLLPTEMGFKVS

SEQ ID NO: 13 - protein, 37 aa, Homo sapiens
ACDTATCVTHRLAGLLSRSGGVVKNNFVPTNVGSKAF

SEQ ID NO: 14 - protein, 13 aa, synthetic construct
SSSHPVFHRGEFS

SEQ ID NO: 15 - protein, 6 aa, synthetic construct
DSVSVW

SEQ ID NO: 16 - protein, 6 aa, synthetic construct
TDIKGK

SEQ ID NO: 17 - protein, 8 aa, synthetic construct
YFFETKCR

SEQ ID NO: 18 - protein, 8 aa, synthetic construct
YCTTTHTF

SEQ ID NO: 19 - protein, 9 aa, synthetic construct
RIDTACVCV

SEQ ID NO: 20 - protein, 7 aa, synthetic construct
RGIDSKH

SEQ ID NO: 21 - protein, 13 aa, synthetic construct
SSSHPIFHRGEFS

SEQ ID NO: 22 - DNA, 6208 bp, synthetic construct
  source 1..6208: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagaggatcgcatcaccatcaccatcacggtagcggatcggaaaacctgtattttcagggatccatgtctatgctgttctacaccctgatcaccgctctgctgatcggtatccgtgctgaaccgcacccggaatctcacgttccggctggtcacgctatcccgcacgctcactggaccaaactgcagcactctctggacaccgctctgcgtcgtgctcgttctgctccggctggtgctatcgctgctcgtgttaccggtcagacccgtaacatcaccgttgacccgaaactgttcaaaaaacgtcgtctgcgttctccgcgtgttctgttctctacccacccgccgccggttgctgctgacgctcaggacctggacctggaagctggttctaccgcttctgttaaccgtacccaccgttctaaacgttcttcttctcacccggttttccaccgtggtgaattctctgtttgcgactctgtttctgtttgggttggtgacaaaaccaccgctaccgacatcaaaggtaaagaagttatggttctgggtgaagttaacatcaacaactctgttttcaaacagtacttcttcgaaaccaaatgccgtgacccgaccccggttgactctggttgccgtggtatcgactctaaacactggaactcttactgcaccaccacccacaccttcgttaaagctctgaccatggacggtaaacaggctgcgtggaggttcatccgtatcgacaccgcttgcgtttgcgttctgtctcgtaaagctggtcgtcgtgctggttctggttctcgtccgaaaccgcagcagttcttcggtctgatgtaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 23 - DNA, 5845 bp, synthetic construct
  source 1..5845: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagaggatcgcatcaccatcaccatcacggtagcggatcggaaaacctgtattttcagggatcctcttcttctcacccggttttccaccgtggtgaattctctgtttgcgactctgtttctgtttgggttggtgacaaaaccaccgctaccgacatcaaaggtaaagaagttatggttctgggtgaagttaacatcaacaactctgttttcaaacagtacttcttcgaaaccaaatgccgtgacccgaccccggttgactctggttgccgtggtatcgactctaaacactggaactcttactgcaccaccacccacaccttcgttaaagctctgaccatggacggtaaacaggctgcgtggaggttcatccgtatcgacaccgcttgcgtttgcgttctgtctcgtaaagctggtcgtcgtgctggttctggttctcgtccgaaaccgcagcagttcttcggtctgatgtaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 24 - DNA, 6106 bp, synthetic construct
  source 1..6106: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagaggatcgcatcaccatcaccatcacggtagcggatcggaaaacctgtattttcagggatccatgcgttctgctccggctggtgctatcgctgctcgtgttcgtccgaaaccgcagcagttcttcggtctgatgatcaccgttgacccgaaactgcgtccgaaaccgcagcagttcttcggtctgatgcgttctccgcgtgttctgttctctacccacccgccgccggttgctgctgaccgtccgaaaccgcagcagttcttcggtctgatgcgttcttcttctcacccggttttccaccgtccgaaaccgcagcagttcttcggtctgatgggtgaattctctgtttgcgactctgtttctgtttgggttcgtccgaaaccgcagcagttcttcggtctgatgaaagaagttatggttctgggtgaacgtccgaaaccgcagcagttcttcggtctgatggttttcaaacagtacttcttcgaacgtccgaaaccgcagcagttcttcggtctgatgcgtgacccgaccccggttgactctggttgccgtggtcgtccgaaaccgcagcagttcttcggtctgatgtggaactcttactgcaccaccacccacaccttcgttaaagctcgtccgaaaccgcagcagttcttcggtctgatgcgtatcgacaccgcttgcgtttgcgttctgtctcgtccgaaaccgcagcagttcttcggtctgatgtaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 25 - DNA, 5989 bp, synthetic construct
  source 1..5989: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagaggatcgcatcaccatcaccatcacggtagcggatcggaaaacctgtattttcagggatccatgagcagttcacatcccgtatttcacaggggcgagttcagcgtgtgcgactctgtgagcgtgtgggttggtgataaaaccacggctaccgacatcaaaggtaaggaggtgatggttttgggcgaagttaatattaacaactccgtctttaaacaatatttcttcgaaaccaagtgccgtgatccgactccggttgattcggggtgccgcggtattgactccaaacattggaattcctactgcaccactacgcacacctttgtaaaggctctgaccatggatggtaagcaggcagcgtggcgttttatccgcatcgacaccgcgtgtgtttgcgtgctctctcgtaaagcgggccgtcgtgccagctgtaataccgcgacctgtgttacccaccgcctggcaggcctgctgagccgtagcggtggtgtcgtgaagaacaacttcgttccgacgaacgtgggcagcgaggcgttttaaggatccggcagctggagccacccgcagttcgaaaagtaaaaaaggatcttggcgtcgacctgcagccaagccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 26 - protein, 278 aa, synthetic construct
MRGSHHHHHHGSGSENLYFQGSMSMLFYTLITALLIGIRAEPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGSGSRPKPQQFFGLM

SEQ ID NO: 27 - protein, 157 aa, synthetic construct
MRGSHHHHHHGSGSENLYFQGSSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGSGSRPKPQQFFGLM

SEQ ID NO: 28 - protein, 244 aa, synthetic construct
MRGSHHHHHHGSGSENLYFQGSMRSAPAGAIAARVRPKPQQFFGLMITVDPKLRPKPQQFFGLMRSPRVLFSTHPPPVAADRPKPQQFFGLMRSSSHPVFHRPKPQQFFGLMGEFSVCDSVSVWVRPKPQQFFGLMKEVMVLGERPKPQQFFGLMVFKQYFFERPKPQQFFGLMRDPTPVDSGCRGRPKPQQFFGLMWNSYCTTTHTFVKARPKPQQFFGLMRIDTACVCVLSRPKPQQFFGLM

SEQ ID NO: 29 - protein, 180 aa, synthetic construct
MRGSHHHHHHGSGSENLYFQGSMSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRASCNTATCVTHRLAGLLSRSGGVVKNNFVPTNVGSEAF

(19) United States
(12) Patent Application Publication
(10) Pub. No.: US 2025/0099561 A1
(43) Pub. Date: Mar. 27, 2025

(54) ACTIVE IMMUNIZATION FOR REDUCING OSTEOARTHRITIC, NEUROPATHIC, AND CANCER PAIN

(71) Applicants: Luis Hector BARBEITO ERBA, Montevideo (UY); Institut Pasteur de Montevideo, Montevideo (UY)

(72) Inventors: Luis Hector BARBEITO ERBA, Montevideo (UY); Emiliano TRIAS TEJERIA, Montevideo (UY); Valentina Varela PIEDRA BUENA, Montevideo (UY); Gabriel Gaston SEMIGLIA REPETTO, Montevideo (UY); Andrea Elena FILOMENO ANDRIOLO, Montevideo (UY); Carolina SEMIGLIA ALVAREZ, Montevideo (UY)

(21) Appl. No.: 18/730,908

(22) PCT Filed: Jan. 20, 2023
(86) PCT No.: PCT/IB2023/050517

§ 371 Date: Jul. 22, 2024

Related U.S. Application Data
(60) Provisional application No. 63/301,873, filed on Jan. 21, 2022.

(51) Int. Cl. A61K 39/00; A61P 25/04; A61P 37/04; C07K 14/48; C07K 14/585

(52) U.S. Cl. CPC A61K 39/0007; A61P 25/04; A61P 37/04; C07K 14/48; C07K 14/585; A61K 2039/54; A61K 2039/545; A61K 2039/552; A61K 2039/55566; A61K 2039/575; C07K 2319/40

(57) ABSTRACT

A recombinant fusion protein used for active immunization or vaccine in the treatment of pain in a subject and a method thereof. The recombinant fusion protein includes: a nerve growth factor (NGF); and substance P (SP) or a calcitonin gene-related peptide (CGRP). The pain can be associated with osteoarthritis (OA), neurogenic inflammation, neuropathy, rheumatoid arthritis, post-surgery or cancer. The invention is particularly useful for treating OA pain in animals.

This application claims priority to U.S. Provisional Application No. 63/301,873, filed Jan. 21, 2022, the contents of which are incorporated herein by reference in their entirety.

### BACKGROUND

### Field of the Invention

The invention relates to vaccine technology, immunotherapy, veterinary and medicine. The invention relates to recombinant fusion proteins and immunogenic compositions, methods for engineering and producing the recombinant fusion proteins and their applications for immunotherapy to diseases involving inflammation and/or pain in mammals, including canine, feline, equine and human. The invention relates to the use of recombinant fusion proteins and immunogenic compositions in active immunization or vaccination and in methods for treating or preventing nociceptive-associated pain and/or inflammatory-associated pain, in particular osteoarthritic, neuropathic, nociceptive and cancer-related pain.

### Discussion of the Related Art

Osteoarthritis (OA) is the most common cause of chronic pain in humans and companion animals. It affects up to 80% older dogs and is a major cause of euthanasia due to major deterioration of quality of life. OA is a chronic, non-curable, degenerative disease affecting moving joints, leading to motor disability. Medical management is complex and multimodal, and focuses on functional and pharmacological treatments to slow its progression and alleviate pain. The presence of pain is often assumed because of motor claudication and improved function following treatment with anti-inflammatory drugs. In the absence of a cure, the primary therapeutic goal is to alleviate the pain through pharmacological or immunological methods. Nonsteroidal anti-inflammatory drugs (NSAIDs) are only partially effective and do not provide complete pain relief in dogs with OA. Moreover, ongoing treatments often have adverse effects, including serious gastro-intestinal and kidney toxicity. When NSAIDs are ineffective or poorly tolerated, adjunctive drugs such as corticosteroids or opioid analgesics may be indicated to relieve OA-associated signs of moderate or severe pain. Unfortunately, the failure of conventional medications results in diminished quality of life, chronic pain, and suffering, which often leads to euthanasia.

The nerve growth factor (NGF) is a neuropeptide which was originally identified as a critical factor for the development and maintenance of sensory and sympathetic neurons in the developing nervous system and later found to have a role in inflammatory hyperalgesia. NGF is the founding member of the neurotrophins, a family of secreted growth factors responsible for the growth, survival, and developmental plasticity of neuronal populations in the vertebrate peripheral and central nervous system. NGF has also been shown to play a key role in the generation of acute and chronic pain and in hyperalgesia in diverse pain states. NGF is expressed at high levels in damaged or inflamed tissues and facilitates pain transmission by nociceptive neurons through a variety of mechanisms.

In the adult system, NGF has an important role in pro-nociception via the NGF-specific tyrosine kinase receptor (TrkA). In OA, NGF is upregulated and secreted by inflammatory cells, fibroblasts, and synoviocytes. Activation of high-affinity NGF-specific TrkA receptors located in sensory fibers results in increased excitability and post-translational changes in the transient receptor potential vanilloid receptor 1 (TRPV1) cation channel. NGF released from inflamed tissues also activates infiltrating mast cells, which in turn further sensitize sensory neurons through secretion of various inflammatory factors. In patients with OA whose tissues are inflamed, NGF level tends to be higher than normal. Blockade of NGF signaling by neutralization of extracellular NGF using specific antibodies have proven to be an effective anti-nociceptive approach.

Recently, attention has been focused on monoclonal antibodies (mAb) that neutralize NGF activity to reduce hyperalgesia and behavioral indicators of pain in various animal models of inflammatory arthritis and patients with OA. Administration of anti-NGF antibodies in passive immunization are effective in reducing pain in animal models of arthritis pain. A canine-specific mAb against NGF showed clinical efficacy in alleviating signs of pain in dogs with osteoarthritis. In human clinical studies, several anti-NGF mAbs have shown to reduce pain and improve function in patients with OA. One anti-NGF monoclonal antibody, Tanezumab, is currently under review by the Food and Drug Administration as a possible treatment for moderate-to-severe OA. In a recent study of OA patients receiving injections of 5 mg/dose of tanezumab, significant improvement in relieving pain and improving physical function were observed. However, about 6% of the patients enrolled in the study experienced adverse effects such as rapid progression of the disease. Although NGF-blocking antibodies appear promising, the dosing required to reduce chronic arthritis pain is associated with safety concerns resulting in a narrow therapeutic index.

Substance P (SP) is a decapeptide belonging to the tachykinin neuropeptide family, which also includes neurokinins A and B. All three peptides share a common C terminal sequence Phe-X-Gly-Leu-Met-NH2. SP is synthesized in the cell bodies of peripheral sensory neurons located in the dorsal root ganglia and exported to the nerve terminals by a fast-axonal transport system. SP is best known as a sensory neurotransmitter mediating nociception in central sensory afferents. SP is released at the peripheral nerve terminals from small unmyelinated and myelinated efferent sensory fibers, causing neurogenic inflammation through the stimulation of cytokine release by various cell types including macrophages and mast cells. Neurogenic inflammation is characterized by local vasodilation, increased vascular permeability, and local immune response. In this context, SP contributes to the development of inflammatory pain through simultaneous activity on central sensitization and associated hyperalgesia, as well as the peripheral tissues through neurogenic inflammation. Compound CP-96,345, which is an antagonist of the SP receptor NK1-R exerts anti-nociception in rat models of inflammatory pain. In comparison, other NK1-R antagonists such as the clinically approved anti-emetic drug Maropitant failed to exert significant antinociceptive activity in different pain conditions. Because of the intrinsic biological complexity of neurokinins transmitters and their receptors, it has been difficult to develop clinically effective small drugs targeting SP receptors for the treatment of pain.

SP plays an important role in the development of arthritis as evidenced by a positive correlation between the size and severity of joint destructive changes. SP levels and its receptor NK1-R expression are increased in the synovial fluid obtained from rheumatoid arthritis patients, which actions on synovial cells lead to cartilage and bone damage. Similarly, SP concentration in patients with OA is positively correlated with the intensity of chronic pain, further suggesting that SP greatly contributes to inflammation and pain associated with osteoarticular damage. In animal models of OA, SP and NK receptors have been linked to joint pain, inflammation, and injury. It has been shown that SP receptor antagonists can help reduce arthritis pain and swelling, although these results have not been confirmed in other studies using NK1-R antagonists. Moreover, evidence have been provided in a pre-clinical study for SP (locally applied as a self-assembled peptide) being protective in OA. As a neuropeptide, it functions as a neurotransmitter and neuromodulator. Substantial evidence supports the role of substance P in promoting pain. The release of SP from immune cells, smooth muscle, blood vessels, and other cells in the periphery, promotes neurogenic inflammation.

Calcitonin gene-related peptide (CGRP) which is a 37-amino acid neuropeptide and its receptors are implicated in nociceptive pathways in peripheral and central nervous system. CGRP is a highly potent vasodilator and, partly therefore, possesses protective mechanisms that are important for physiological and pathological conditions involving the cardiovascular system and wound healing. CGRP is primarily released from sensory nerves and thus is implicated in pain pathways.

To date, no studies have evaluated the efficacy of adjunctive anti-nociceptive therapeutics in the context of active immunization or immunotherapy for dogs which aim at reducing refractory OA pain while maintaining satisfactory safety profiles.

### SUMMARY OF THE INVENTION

OA is a prevalent disease condition in dogs leading to refractory pain and functional disability, frequently resulting in euthanasia. The present disclosure evaluates the beneficial effects of systemic active immunization against at least 2 nociceptive mediators such as nerve growth factor (NGF); and Substance P (SP) or CGRP as add-on therapy to mitigate OA-associated pain, thus delaying functional deterioration and euthanasia in domesticated dogs. Active immunization against at least 2 nociceptive mediators such as NGF, SP or CGRP complements conventional treatment currently offered to relieve refractory OA-associated pain, improves dogs' quality of life and spare suffering dogs from euthanasia.

The invention relates to active immunization, vaccine and immunogenic compositions for the treatment of inflammatory, neuropathic and cancer-associated pain, which is mediated by endogenous nociceptive mediators acting in concert. The immunogenic compositions contain sequences of nociceptive mediators expressed as fusion recombinant immunogens from canine, feline, equine, or human including NGF; and SP or CGRP, or fragments thereof. Said immunogens are prepared as recombinant fusion proteins in the form of inclusion bodies which may be further purified using high molarity urea following extraction of undesired bacterial components. The immunogenic compositions elicit an immune response, including antibodies that are directed against two or more endogenous proteins which are preferentially nociceptive mediators. The new vaccine thus has a multi-valent capability against pain pathways and perception and also the capability of producing immunotherapeutic or prophylactic benefit against more than one health hazard. The immunogenic compositions of the invention induce an efficient immune response, in particular trigger the production of antibodies against nociceptive mediators. Advantageously, when administered to mammals, such as dogs, cats, horses or humans, the immunogenic compositions of the invention alleviate nociceptive and/or inflammatory-associated pain and reduce the need for painkillers in chronic and/or refractory pain associated with rheumatoid pain, osteoarticular inflammation, neuropathic lesions or cancer. The immunogenic compositions according to the invention are preferably administered as divalent or multivalent vaccines comprising adjuvant(s).

The invention relates to a method for inducing an immune reaction, encompassing the systemic administration of immunogens subcutaneously or intramuscularly, similar to vaccines used to protect against infectious agents, but inducing the production of antibodies that recognize and neutralize the activity of inflammatory proteins or nociceptive mediators.

The invention relates to antigenic polypeptides produced in genetically modified microorganisms, which are engineered to express amino-acid sequences of at least two inflammatory and/or nociceptive mediators such as NGF, SP and/or CGRP that are known to act in concert in mediating pain at the peripheral or central nervous system. The recombinant immunogenic proteins are preferably produced as inclusion bodies, purified to decrease the levels of microbial endotoxins and used as immunogenic compositions in the context of active immunity.

The invention relates to the design and production of polyvalent recombinant polypeptide antigens, which when used for active immunization stimulate the production of autoantibodies with neutralizing activity to the referred inflammatory and/or nociceptive mediators.

The invention relates to the recombinant polypeptides containing two or more immunogenic fragments of nociceptive mediators that are obtained in the form of inclusion bodies following extraction of bacterial endotoxins.

The invention relates to immunogen or immunogenic protein production by recombinant fusion protein technologies with the ability to elicit specific immune response in a subject. Such vaccines are particularly designed for the treatment of diseases involving chronic inflammation and osteoarthritis pain, nociceptive pain, neuropathic pain, and cancer pain in mammals including canines, felines, equines and humans.

The invention relates to the production and use of antigenic recombinant fusion proteins for the treatment and/or prevention of mast cells-, neutrophils-, osteoclasts- or nociceptors-related disorders, particularly inflammatory pain, in pets and farm animals.

The invention relates to recombinant fusion proteins for treating pain caused by osteoarthritis in a subject. The recombinant fusion proteins include NGF; and SP or CGRP.

Further aspects are provided by the subject matter of the following embodiments.

A recombinant fusion protein for treating pain in a subject having: at least one immunogenic fragment derived from a nerve growth factor (NGF); and at least one immunogenic fragment derived from substance P (SP) or calcitonin gene-related peptide (CGRP), wherein the recombinant fusion protein elicits the production of neutralizing antibodies.

The recombinant fusion protein of the preceding embodiment, wherein the neutralizing antibodies are neutralizing antibodies against NGF.

The recombinant fusion protein of any of the preceding embodiments, wherein the neutralizing antibodies are neutralizing antibodies against SP or CGRP.

The recombinant fusion protein of any of the preceding embodiments, including an amino acid sequence having at least 90%, at least 95% or 100% sequence identity with SEQ ID NO: 26, SEQ ID NO: 27, SEQ ID NO: 28 or SEQ ID NO: 29.

The recombinant fusion protein of any of the preceding embodiments, including an NGF amino acid sequence having at least 90%, at least 95% or 100% sequence identity with SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

The recombinant fusion protein of the preceding embodiment, wherein the NGF amino acid sequence includes at least one immunogenic fragment having at least 6 consecutive amino acids of SEQ ID NO: 1 or SEQ ID NO: 5.

The recombinant fusion protein of any of the preceding embodiments, wherein the at least one immunogenic fragment acid has an amino acid sequence having at least 90%, at least 95% or 100% sequence identity with SEQ ID NO: 14, SEQ ID NO: 15, SEQ ID NO: 16, SEQ ID NO: 17, SEQ ID NO: 18, SEQ ID NO: 19 or SEQ ID NO: 20.

The recombinant fusion protein of any of the preceding embodiments, wherein the NGF amino acid sequence includes at least one immunogenic fragment having at least 6 consecutive amino acids of SEQ ID NO: 4 or SEQ ID NO: 8.

The recombinant fusion protein of any of the preceding embodiments, wherein the at least one immunogenic fragment has an amino acid sequence having at least 90%, at least 95% or 100% sequence identity with SEQ ID NO: 21.

The recombinant fusion protein of any of the preceding embodiments, comprising an SP amino acid sequence having at least 90%, at least 95% or 100% sequence identity with SEQ ID NO: 9.

The recombinant fusion protein of the preceding embodiments, wherein the SP amino acid sequence includes at least one immunogenic fragment having at least 6 consecutive amino acids of SEQ ID NO: 9.

The recombinant fusion protein of any of the preceding embodiments, having a CGRP amino acid sequence with at least 90%, at least 95% or 100% sequence identity with SEQ ID NO: 10, SEQ ID NO: 11, SEQ ID NO: 12 or SEQ ID NO: 13.

The recombinant fusion protein of any of the preceding embodiments, wherein the CGRP amino acid sequence comprises at least one immunogenic fragment having at least 6 consecutive amino acids of SEQ ID NO: 10, SEQ ID NO: 11, SEQ ID NO: 12 or SEQ ID NO: 13.

The recombinant fusion protein comprising SEQ ID NO: 27.

The recombinant fusion protein comprising SEQ ID NO: 29.

A nucleic acid encoding the recombinant fusion protein including NGF; and SP or CGRP of any of the preceding embodiments.

A recombinant vector having at least a nucleic acid sequence encoding the recombinant fusion protein of any of the preceding embodiments.

The recombinant vector of the preceding embodiment, including a nucleic acid sequence having at least 90%, at least 95% or at least 99% sequence identity with SEQ ID NO: 22, SEQ ID NO: 23, SEQ ID NO: 24 or SEQ ID NO: 25.

A method for producing the recombinant fusion protein of any of the preceding embodiments, wherein the recombinant fusion protein is produced in a procaryotic or eukaryotic expression system as an inclusion body.

The method of the preceding embodiment, wherein said recombinant fusion protein is further purified with urea of high molarity ranging from 4 to 8 M.

The method of any of the preceding embodiments, wherein the recombinant fusion protein is expressed from a recombinant vector.

The method of the preceding embodiment, wherein the recombinant vector is the vector of any of in the preceding embodiments.

An immunogenic composition including at least one recombinant fusion protein of any one of the preceding embodiments.

An immunogenic composition produced according to the method of any of the preceding embodiments.

The immunogenic composition of any of the preceding embodiments, further including an acceptable carrier and/or an adjuvant selected from the group consisting of oil-in-water adjuvant, polymer and water adjuvant, water-in-oil adjuvant, aluminum hydroxide adjuvant and combinations thereof.

The immunogenic composition of the preceding embodiment, wherein the adjuvant is the complete or incomplete Freund's adjuvant, a Montanide™, for example Montanide™ Gel (from Seppic), aluminum salts (alum), oil emulsions, saponins, immune-stimulating complexes (ISCOMs), liposomes, microparticles, nonionic block copolymers, derivatized polysaccharides, cytokines, or bacterial derivatives.

The immunogenic composition of any of the preceding embodiments for use as a medicament, preferably a vaccine.

The immunogenic composition of any of the preceding embodiments for use in the treatment and/or the prevention of nociceptive and/or inflammatory-related pain, preferably osteoarthritis (OA)-associated pain, most preferably chronic and/or refractory OA-related pain.

The immunogenic composition for use according to any of the preceding embodiments wherein the pain is refractory to at least one anti-inflammatory compound selected from the group consisting of a NSAID, a corticosteroid and an opioid analgesic.

A pharmaceutical composition for treating or preventing pain in a subject having at least one recombinant fusion protein of any of the preceding embodiments and a pharmaceutically acceptable carrier. The pharmaceutical composition includes the recombinant fusion protein having NGF; and SP or CGRP; and a pharmaceutically acceptable carrier.

The pharmaceutical composition of the preceding embodiment, wherein the pain is associated with OA, neurogenic inflammation, neuropathy, rheumatoid arthritis, post-surgery or cancer.

The pharmaceutical composition of any of the preceding embodiments, wherein the pain is a nociceptive and/or inflammatory-related pain, preferably (OA)-associated pain, most preferably chronic and/or refractory OA-related pain.

The pharmaceutical composition of the preceding embodiment, wherein the pain is refractory to at least one anti-inflammatory compound selected from the group consisting of a NSAID, a corticosteroid and an opioid analgesic.

A method for treating or preventing pain in a mammal by administering an effective amount of the immunogenic composition of any of the preceding embodiments.

The method of the preceding embodiment, wherein the pain is associated with OA, neurogenic inflammation, neuropathies, rheumatoid arthritis, post-surgery or cancer.

The method of the preceding embodiment for treating pain caused by osteoarthritis in a subject includes the step of administering to the subject a therapeutically effective amount of the recombinant fusion protein including NGF; and SP or CGRP.

The method of any of the preceding embodiments, wherein the pain is a nociceptive and/or inflammatory-related pain, preferably OA-associated pain, most preferably chronic and/or refractory OA-related pain.

The method of the preceding embodiment, wherein the pain is refractory to at least one anti-inflammatory compound selected from the group consisting of a NSAID, a corticosteroid and an opioid analgesic.

The method of any of the preceding embodiments, wherein the immunogenic composition is administered in combination with at least one neutralizing antibody directed against at least one nociceptive mediator including NGF, SP and CGRP.

The method of the preceding embodiment, wherein the at least one neutralizing antibody is directed against NGF and is selected from the group consisting of tanezumab, fasinumab and fulranumab.

The method of any of the preceding embodiments, wherein the immunogenic composition is administered in combination with at least one receptor antagonist that blocks a nociceptive signaling pathway, preferably a neurokinin-1 receptor (NK1-R) antagonist, most preferably compound CP-96,345.

The method of any of the preceding embodiments, wherein the immunogenic composition is administered in a combination therapy with at least one anti-inflammatory compound selected from the group consisting of NSAID, a corticosteroid and an opioid analgesic.

The method of any of the preceding embodiments, wherein the immunogenic composition is administered orally, subcutaneously, intramuscularly or transdermally, preferentially subcutaneously.

The method of any of the preceding embodiments, wherein the first immunization is followed by a booster dose after about 2 weeks, preferably by 3 booster doses each administered about 2 weeks apart.

The method of any of the preceding embodiments, wherein the mammal is a canine, human, feline, or equine.

The method of any of the preceding embodiments, wherein the mobility and/or functional activities (e.g., walking, running, climbing stairs, rising to a standing position) improve and/or the intensity of the pain is reduced in the treated mammal.

A vaccine for treating or preventing OA-associated pain in dogs that includes the NGF-SP recombinant fusion protein of amino acid sequence SEQ ID NO: 27.

A vaccine for treating or preventing OA-associated pain in dogs that includes the NGF-CGRP recombinant fusion protein of amino acid sequence SEQ ID NO: 29.

A method for treating or preventing OA-associated pain in dogs that includes the subcutaneous administering of a vaccine that contains the NGF-SP recombinant fusion protein having the amino acid sequence SEQ ID NO: 27 or the NGF-CGRP recombinant fusion protein having the amino acid sequence SEQ ID NO: 29 followed by 3 booster injections administered approximately 2 weeks apart.

### BRIEF DESCRIPTION OF THE DRAWINGS

FIG. 1 is a schematic representation showing the design, production, and immunization with Xep01 (NGF-SP) in osteoarthritis dogs suffering from chronic refractory pain.

FIGS. 2A-2D show the immune response elicited after immunization of mice with recombinant fusion proteins NGF-SP and NGF-CGRP. FIG. 2A shows a mass spectrometry analysis of a recombinant fusion immunogen, NGF-SP. FIG. 2B shows a mass spectrometry analysis of a recombinant fusion immunogen, NGF-CGRP.

FIG. 2C is a schematic representation showing a mice immunization protocol. FIG. 2D is a graphical representation showing the production of antibody titers against NGF and SP after immunization with the NGF-SP recombinant fusion protein. FIG. 2E is a graphical representation showing the production of antibody titers against NGF and CGRP after immunization with the NGF-CGRP recombinant fusion protein.

FIGS. 3A-3C show antibody titers after immunization with a recombinant fusion immunogen NGF-SP eliciting an immune response in horses. FIG. 3A is a schematic representation of the immunization protocol. FIG. 3B is a graphical representation showing the production of antibody titers against NGF. FIG. 3C is a graphical representation showing the production of antibody titers against SP.

FIGS. 4A-4C show the antibody titers after immunization with a recombinant fusion immunogen NGF-SP eliciting an immune response in dogs. FIG. 4A is a schematic representation of the immunization protocol. FIG. 4B is a graphical representation showing the production of antibody titers against NGF. FIG. 4C is a graphical representation showing the production of antibody titers against SP.

FIGS. 5A-5D show a description of pain according to the dogs' owners Canine Brief Pain Inventory (CBPI). FIG. 5A is a graphical representation showing CBPI scores regarding the worst pain. FIG. 5B is a graphical representation showing CBPI scores regarding the least pain. FIG. 5C is a graphical representation showing CBPI scores regarding average pain. FIG. 5D is a graphical representation showing CBPI scores regarding pain at the moment of veterinarian revision.

FIGS. 6A-6F show description of function according to the dogs' owners CBPI. FIG. 6A is a graphical representation showing CBPI scores regarding general activity. FIG. 6B is a graphical representation showing CBPI scores regarding the enjoyment of life. FIG. 6C is a graphical representation showing CBPI scores regarding the ability to rise to standing from lying down. FIG. 6D is a graphical representation showing CBPI scores regarding the ability to walk. FIG. 6E is a graphical representation showing CBPI scores regarding the ability to run. FIG. 6F is a graphical representation showing CBPI scores regarding the ability to climb stairs.

FIG. 7 is a graphical representation showing the effect of NGF-SP vaccination on the pain severity score (PSS) and the pain interference score (PIS).

FIGS. 8A-8C show active immunization with NGF-SP improves clinical outcomes assessed by veterinarians following the Colorado State University Canine Acute Pain Scale. FIG. 8A is a graphical representation showing clinical scores regarding the pain on palpation. FIG. 8B is a graphical representation showing clinical scores regarding walking/lameness. FIG. 8C is a graphical representation showing clinical scores regarding general activity.

FIGS. 9A-9C show pain medication consumption in osteoarthritis dogs followed during and after immunization protocol. FIG. 9A is a graphical representation showing percentages of dogs treated with pain medications. FIG. 9B is a graphical representation showing percentages of dogs treated with NSAIDs. FIG. 9C is a graphical representation showing percentages of dogs treated with corticosteroids.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be use and other methods developed without departing from the broad concepts of the current invention. All references cited anywhere in this specification, including the Background and Detailed Description sections, are incorporated by reference as if each had been individually incorporated.

Definitions are included herein for the purpose of understanding the present subject matter and the appended claims. The abbreviations used herein have their conventional meanings within the chemical and biological arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994): Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The present description identifies certain nucleotide and amino acid sequences (polynucleotides and polypeptides) as part of the invention. It is to be understood that the specifically identified sequences adequately describe other sequences that contain less than 100% sequence identity but to the identified sequences that provide the same function. For example, a nucleotide sequence may have 90% sequence identity or 95% sequence identity with a polynucleotide specifically disclosed herein and still encode for an entirely equivalent or functionally equivalent polypeptide. Similarly, a polypeptide may contain less than 100% sequence identity to a polypeptide specifically identified herein and provide the same function. For example, a polypeptide may have 90% sequence identity or 95% sequence identity with a polypeptide specifically disclosed herein and still retain the same or sufficiently similar activity or functionality as the specifically identified polypeptide.

As used throughout, the term “gene” refers to a nucleic acid sequence or a part thereof having a functional role in protein coding or transcription, or regulation of other gene expression. The gene may be composed of all nucleic acids encoding a functional protein or a part of the nucleic acid encoding or expressing the protein. The nucleic acid sequence may include a gene mutation in exon, intron, initiation or termination region, promoter sequence, other regulatory sequence, or a unique sequence adjacent to the gene.

As used throughout, the term “nociceptive factors or mediators” refers to molecules that directly or indirectly promote an increased peripheral sensitization to stimuli resulting in painful perception, including but not limiting to NGF, SP and CGRP. Nociceptive mediators activate primary afferent neurons directly or indirectly to enhance nociceptive signal transmission to the central nervous system. Excitation of primary afferents by peripherally originating mediators, so-called “peripheral sensitization” is a hallmark of tissue injury-related pain. For example, chronic OA pain is associated with sensory disturbances that are mainly mediated by endogenous nociceptive factors produced in OA-damaged tissues. Such nociceptive mediators may also exhibit inflammatory activities. NGF and SP functionally crosstalk in inflammation and pain perception. In OA, NGF produced by inflammatory cells induces the overexpression SP in sensory neurons, which in turn contributes to hyperalgesia and neurogenic inflammation at peripheral sites. SP and NGF are recognized inflammatory mediators produced by immune cells with the ability to promote mast cell degranulation.

As used throughout, the term “immunogenic fragment or immunogen” refers to an amino acid sequence that has the ability to induce a humoral and/or cell-mediated immune response. For example, an immunogenic fragment of the NGF polypeptide is capable of eliciting the production of antibodies against NGF.

As used throughout, the term “antibody or immunoglobulin” refers to a protein produced by the B-cells of the immune system that can identify, bind and neutralize an antigen. In the context of the invention, an antibody is produced by the immune system and binds an endogenous protein or polypeptide, such as a nociceptive mediator. The antibody may have neutralizing properties and may be capable of suppressing or reducing the biological activity of the nociceptive mediator or the downstream pathway mediated by the nociceptive mediator such as blocking its binding to its specific receptor.

As used throughout, the term “active immunization” refers to immunization that stimulates the immune system to produce antibodies against an antigen (self or foreign). Active immunization can be induced through vaccination. In the context of the invention, a vaccine or immunogenic composition comprises at least 2 self-antigens or endogenous polypeptides that stimulate the production of antibodies without causing any illness. Such antibodies may have neutralizing properties that will capture the self-antigens, blocking any of their function(s). Active immunization is often long-lasting and may be reactivated by repeated injection of boosters. In contrast, passive immunization occurs when antibodies directed against specific antigen are administered to a subject.

As used throughout, the term “adjuvant” refers to a substance that increases the intensity of the immune response after co-administration with an immunogen. An adjuvant may act as an immunopotentiator useful for enabling immunogenic composition or vaccine to induce potent and persistent immune responses, while reducing the dose and number of boosters. Adjuvant may also increase the stability of the immunogenic composition or vaccine.

As used throughout, the term “osteoarthritis (OA)” refers to a chronic joint disease characterized by loss of joint cartilage, thickening of the joint capsule and new bone formation around the joint (osteophytosis) and ultimately leading to pain and limb dysfunction. In canines, signs of OA are often non-specific and include: i) activity impairment, reluctance to exercise, decrease in overall activity, stiffness, lameness, inability to jump, changes in gait such as “bunny-hopping”, ii) pain on manipulation, behavioral changes such as aggression or signs of discomfort.

As used throughout, the term “refractory osteoarthritis” refers to a chronic condition that does not respond or only slightly responds to conventional treatment including NSAIDs, corticoids and opioids.

As used throughout, the term “neurogenic inflammation” refers to the physiological process by which mediators are released directly from the sensory nerves to initiate an inflammatory reaction. This results in production of local inflammatory responses including erythema, swelling, temperature increase, tenderness, and pain. Fine unmyelinated afferent somatic C-fibers, which respond to low intensity mechanical and chemical stimulations, are largely responsible for the release of inflammatory mediators. When stimulated, these nerve fibers in the cutaneous nerves rapidly release active neuropeptides such as SP and CGRP into the microenvironment, triggering a series of inflammatory responses.

As used throughout, the term “inflammatory pain” refers to the spontaneous hypersensitivity to pain that occurs in response to tissue damage and inflammation (e.g., postoperative pain, trauma, arthritis). Inflammatory pain is a type of nociceptive pain that results from activation and sensitization of nociceptors by inflammatory mediators. Often the pain improves when the inflammation subsides.

As used throughout, the term “chronic pain” refers to pain that is ongoing and usually lasts longer than six months. Chronic pain is not simply a temporal continuum of acute pain. In the setting of persistent injury, functional and structural reorganization of neuronal circuits in the CNS leads to long-term changes in perception and behavior. Such pain can persist after an injury or illness with pain signals remaining active in the nervous system for weeks, months or years.

As used throughout, the term “refractory pain” refers to pain that cannot be alleviated with conventional painkillers including anti-inflammatory compounds such as NSAIDs, corticosteroids and opioid analgesics.

As used throughout, the term “cancer” refers to a disease characterized by the rapid and uncontrolled growth of aberrant cells. Cancer patients may experience nociceptive pain caused by the cancer per se, when tumors grow larger causing damage to the surrounding tissue.

As used throughout, the term “self-antigen” refers to any molecule or chemical group of an organism which acts as an antigen in inducing antibody production in another organism but to which the healthy immune system of the parent organism is tolerant. Immunization/vaccine against self-antigens requires a specific design of the immunogens and formulations that allows the vaccination to break the self-tolerance in a specific organism. Due to several central and peripheral tolerance mechanisms, it is extremely challenging to induce an immune response to self-antigens. In the context of the invention, the self-antigens are nociceptive mediators such as NGF, SP and CGRP.

As used throughout, the term “recombinant protein” refers to a protein encoded by recombinant nucleic acid that has been cloned in an expression vector that supports expression of the gene and translation of messenger RNA. Escherichia coli (bacteria) is one of the organisms of choice for the production of recombinant proteins. Its use as a cell factory is well-established and it has become the most popular expression platform. High-level expression of many recombinant proteins in Escherichia coli leads to the formation of highly aggregated protein commonly referred to as inclusion bodies. Inclusion bodies are normally formed in the cytoplasm. Bacterial inclusion bodies are mesoscale protein aggregates commonly observed in recombinant bacteria, primarily formed by recombinant protein. Other expression system may include but are not limited to insect cells and yeast cells.

As used throughout, the term “fusion protein or chimeric protein” refers to a hybrid protein or polypeptide having an amino acid sequence comprising at least two partial or complete sequences derived from, obtained from, or isolated from different polypeptides that are not naturally adjoined. The terms “fusion” and “chimeric” are used interchangeably throughout. A fusion protein or fusion polypeptide is the functional product of a fusion gene or fusion nucleic acid sequence. Fusion gene can further be modified by mutation, deletion, insertion or substitution of heterologous sequences, or by any means available using recombinant DNA technology.

As used throughout, the term “fragment” refers to a peptide or polypeptide of chain-type polymer formed by at least 6 amino acid residues which are linked to each other via peptide bonds. It may also include the complete amino acid sequence of the native polypeptide or protein. It may include amino acid sequences that are conservative variations. The terms fragment, peptide and polypeptide are used interchangeably. In the context of the invention, an immunogenic fragment is a peptide or polypeptide capable of eliciting an immune response cellular and/or humoral, including the production of specific antibodies directed to that immunogenic fragment or to the protein having the immunogenic fragment.

The term “conservative variation,” as used herein, denotes the replacement of an amino acid residue by another, biologically similar residue. Examples of conservative variations include substitution of one hydrophobic residue, such as isoleucine, valine, leucine, alanine, cysteine, glycine, phenylalanine, proline, tryptophan, tyrosine, norleucine, or methionine for another, or substitution of one polar residue for another, for example, substitution of arginine for lysine, glutamic acid for aspartic acid, or glutamine for asparagine, and the like. Neutral hydrophilic amino acids which may be substituted for one another include asparagine, glutamine, serine, and threonine. It may also include the use of a substituted amino acid in place of an unsubstituted parent amino acid, provided that an antibody raises against the substituted polypeptide also recognizes the unsubstituted polypeptide. Such conservative substitutions are within the definition of the types of fragments disclosed in the present application.

A person having ordinary skill in the art may make similar substitutions to obtain immunogenic fragments having higher immunogenicity. For example, one aspect disclosed in the present application provides fragments corresponding to amino acid sequences (e.g., SEQ ID NOS: 5-21), as well as analogues, homologs, isomers, derivatives, amidated variations, and conservative variations thereof, as long as the immunogenicity of the fragment remains.

Minor modifications to primary amino acid sequences disclosed in the present application may result in fragments which have substantially equivalent or enhanced immunogenicity, as compared to the specific fragments described herein. Such modifications may be deliberate, as by site-directed mutagenesis, or may be spontaneous.

All peptides, polypeptides or fragments may be synthesized using L-amino acids, but D forms of all of the peptides may be synthetically produced. In addition, C-terminal derivatives, such as C-terminal methyl esters and C-terminal amidates, may be produced in order to increase the immunogenicity of the peptide according to one embodiment disclosed in the present application.

In an embodiment, a recombinant fusion protein NGF-SP includes immunogenic fragments which elicit the production of immunoglobulins that are capable of binding NGF and SP. Similarly, a recombinant fusion protein NGF-CGRP includes immunogenic fragments which elicit the production of immunoglobulins that that are capable of binding NGF and CGRP.

Antibodies possess significant advantages as therapeutic agents because of their higher specificity and reduced off-target effects. By binding to their respective nociceptive mediators, the antibodies may inhibit or reduce the biological activity of the nociceptive mediators and/or their downstream signaling pathways such as blocking the binding to their specific receptor or suppressing the cellular response(s) the mediators induce. Systemic immunization with NGF-SP or NGF-CGRP of subject (e.g., dogs) suffering from severe dysfunction and refractory osteoarthritis results in unexpectedly high titers of anti-NGF and anti-SP or anti-NGF and anti-CGRP serum antibodies, respectively. Such systemic immunotherapy directed to the nociceptive mediators improves the clinical symptoms associated with osteoarthritic pain and significantly reduces the use and/or dose of conventional anti-inflammatory drugs. This is especially important given the severe side effects associated with anti-inflammatory drugs when taken chronically.

While monovalent active or passive immunization to NGF reported in the literature is directed to a single nociceptive mediator such as NGF, the present results provide strong evidence for the synergistic effects of combining NGF and SP or NGF and CGRP immunization in reducing inflammation and pain. Taken together, the results support the concept that systemic immunotherapy targeting at least 2 nociceptive mediators including NGF, and SP or CGRP as therapeutic options for osteoarthritis pain in subjects (e.g., human, equine, canine feline).

The immunogenic composition comprising at least one immunogenic fragment derived from each of at least 2 nociceptive mediators produces synergistic therapeutic effect compared to the effect produced by each mediator individually or their added effects. The concomitant blockade of at least 2 nociceptive mediators results in synergistic analgesic effects, thus decreasing the dosage of antibodies required to obtain an efficacious therapeutic benefit and prevent adverse effects. Surprising and unexpected results show more than an additive effect that would be expected from individual treatment with neutralizing antibodies to NGF and neutralizing antibodies to SP or CGRP. The anti-nociceptive effect of anti-NGF antibodies is potentiated by the concomitant immunological neutralization of SP by anti-SP antibodies or CRGP by anti-CGRP antibodies. The results obtained with the use of inclusion bodies is also unexpected.

Generally, proteins in inclusion bodies have a non-native conformation. The immunogens lack tertiary protein structure and three-dimensional conformational epitopes. However, recombinant fusion proteins administered as inclusion bodies according to the present invention unexpectedly generate neutralizing antibodies to NGF, SP or CGRP.

According to prior art, proteins in inclusion bodies only have linear epitopes that once digested generate small peptides that bind to major histocompatibility complex molecules and then later with T cell receptors through amino acids that are continuous in a line. Furthermore, the prior art teaches that the non-native conformation of proteins upon accumulation in inclusion bodies abrogates their use as vaccines aimed at generating high-affinity or neutralizing antibodies. On the contrary, according to the present invention, inclusion bodies are adequate as an antigenic vaccine formulation.

Advantageously, systemic immunization with inclusion bodies containing bivalent recombinant fusion proteins NGF-SP or NGF-CGRP, are effective in treating pain in subjects (e.g., canines) affected by inflammation-based refractory osteoarthritis. Interestingly, there is not a clear correlation between the long-term efficacy and the drop in the antibody titers 3-4 months after the completion of the immunization protocol. This suggests that other mechanisms of action beyond the blocking capacity of neutralizing antibodies may be at play.

Advantageously, the simultaneous immune response against endogenous NGF, SP or CGRP by antibodies not only decreases osteoarthritis pain but, unexpectedly, leads to long-term mitigation of pain and preserved motor functionality in a way that cannot be predicted from the prior art. This effect was opposed to the effect to that described in osteoarthritis cases in human clinical trials with developmental anti-NGF monoclonal antibodies (tanezumab), where NGF inhibition failed to protect affected joints, also leading to destructive arthropathy and rapidly progressive large joint OA in a small number of patients.

The methods for treating OA and pain associated with inflammation or neuropathic conditions include administering to a subject (e.g., human, mammalian pet or farm animal) one or more immunogenic polypeptides designed and produced as polyvalent recombinant fusion proteins, that stimulate a humoral immunological response in the form of specific antibodies that will simultaneously bind and neutralize the activity of endogenous nociceptive mediators and/or inflammatory enzymes upregulated in the affected individual.

In an embodiment, a method for treating osteoarthritis in an individual includes administering to the individual a polyvalent immunogenic protein that stimulates the production of antibodies upon systemic immunization, wherein the immunogenic protein is an engineered non-natural recombinant fusion protein produced in microbial organism, for example, a recombinant polypeptide containing the sequences or fragments of two or more nociceptive mediators or inflammatory proteins or enzymes.

Surprisingly, systemic immunization with bivalent recombinant fusion proteins as disclosed herein are effective in treating pain affected by osteoarthritis, neuropathic pain or other forms of inflammatory pain. Such a vaccination results in unexpectedly enhanced pain treatment with long-lasting therapeutic effects and few adverse effects. In addition, such therapy generally allows a reduced dosage of NSAIDs or corticoids previously used for clinical management of inflammation and pain.

In an embodiment, a method for treating (or, in other embodiments, preventing) pain includes administering an amount of a multivalent recombinant fusion protein to provide effective pain and inflammation relief. In some embodiments, vaccination will be administered up to 4-6 boosters so as to allow reduction of symptoms.

The methods according to the invention are suitable for treating or preventing any pain of any etiology, including pain where the use of an NSAID is generally prescribed. In some embodiments, the pain is osteoarthritis. In other embodiments, the pain is pain associated with neuropathic pain, rheumatoid arthritis or post-surgical pain.

The vaccine can be administered to an individual via various suitable routes. For example, the vaccine can be administered together or separately, and/or simultaneously and/or sequentially, orally, subcutaneously, intramuscularly or transdermally. In some embodiments, the invention provides a pharmaceutical composition for treating pain comprising an effective amount of divalent- or multivalent vaccine, and a pharmaceutically acceptable carrier.

In an embodiment, a group of recombinant fusion proteins are designed to include at least one immunogenic fragment of each of two or more nociceptive mediators. Recombinant fusion proteins are produced in a genetically modified microorganism. Preferentially, the recombinant fusion proteins have multi-antigenic regions of nociceptive mediators and are obtained in the form inclusion bodies.

In an embodiment, the recombinant fusion protein is obtained as inclusion bodies containing the polypeptides of interest.

In an embodiment, a method for producing a recombinant fusion protein involves the transfection of E. coli cells with an expression vector, the expression of the polypeptide coded by this vector, the isolation of inclusion bodies containing the produced polypeptide used in the production of an immunogen, where the vector contains a nucleotide sequence coding a polypeptide sequence according to the present invention defined above.

The recombinant fusion protein is an immunogen for the treatment of other diseases where inflammation or nociceptive pain are predominant such as osteoarthritis.

The invention provides many advantages over previous therapies known from the state of technology. A very effective and inexpensive method has been devised for inducing the production of antibodies targeting simultaneously two or more nociceptive mediators, which results in an effective clinical therapeutic benefit.

Treatment with human, canine, or feline monoclonal antibodies to NGF have shown clinical efficacy for the treatment of pain in humans, canines, and felines, respectively. (Dietz, Brett W et al.” Rheumatic diseases clinics of North America vol. 47, 2 (2021): 181-195; Enomoto, Masataka et al. The Veterinary record vol. 184, 1 (2019): 23.) Also, systemic immunization to NGF using viral particles in rodents has also shown antinociceptive effects in models of inflammation. However, treatment based on monoclonal antibodies against NGF does not target other inflammatory and nociceptive mediators acting synergistically to mediate pain in osteoarthritis.

Surprisingly, engineered polyvalent recombinant fusion proteins with immunogenic fragments which derived from NGF, SP, CGRP and/or other inflammatory mediators are produced as inclusion bodies. They elicit a humoral immunological response with the concomitant production of neutralizing antibodies directed against the endogenous mediators which synergize under pathological conditions.

Inclusion bodies are readily obtainable and induce strong immune reactions. The immunogenic composition or vaccine produced is easy to administer and lacks the common challenges associated with the preparation and production of monoclonal antibodies, such as the high-cost production.

In an embodiment, the self-antigens are expressed in bacterial cells. The homogeneity of the composition produced guarantees precise dosing by the simple design of the recombinant fusion proteins and further isolation and purification of the inclusion bodies.

The production of polyvalent immunogens by recombinant fusion protein technology with the ability to elicit specific immune response in hosts upon vaccination is a new and simple way to face the problem of invalidating pain occurring in various disease conditions affecting mammalian pets and farm animals.

One aspect of this disclosure provides a recombinant fusion protein for treating pain caused by osteoarthritis in a subject. The recombinant fusion protein comprises: (i) NGF; and (ii) SP or CGRP. The recombinant protein stimulates the production of immunoglobulins against (i) NGF; and (ii) SP or CGRP.

In an embodiment, the recombinant protein is a fusion protein. The fusion protein can be in a form of an inclusion body or bodies. The fusion may be formed via a peptide bond or a chemical bond. The chemical bond can be disulfide bonds, diamine bonds, sulfide-amine bonds, carboxyl-amine bonds, ester bonds, and covalent bonds.

In an embodiment, the subject is a canine, and the recombinant fusion protein has at least 50%, at least 60%, at least 70%, at least 75%, at least 80%, at least 85%, at least 90%, at least 95%, at least 98%, at least 99%, at least 99.5% amino acid sequence identity to SEQ ID NOS: 26, 27, 28, or 29.

In an embodiment, the subject is a canine, and the recombinant fusion protein has an amino acid sequence of SEQ ID NOS: 26, 27, 28, or 29.

In an embodiment, the NGF has at least 50%, at least 60%, at least 70%, at least 75%, at least 80%, at least 85%, at least 90%, at least 95%, at least 98%, at least 99%, at least 99.5% amino acid sequence identity to SEQ ID NOS: 1, 2, 3, 4, 5, 6, 7, or 8.

In an embodiment, the NGF includes a fragment of SEQ ID NO: 1 or SEQ ID NO: 5 and further comprises amino acid sequences of SEQ ID NOS: 14, 15, 16, 17, 18, 19, or 20. In an embodiment, the NGF comprises a fragment of SEQ ID NO: 4 or SEQ ID NO: 8, and further comprises an amino acid sequence of SEQ ID NO: 21.

In an embodiment, the SP has at least 50%, at least 60%, at least 70%, at least 75%, at least 80%, at least 85%, at least 90%, at least 95%, at least 98%, at least 99%, at least 99.5% amino acid sequence identity to SEQ ID NO: 9.

In an embodiment, the CGRP has at least 50%, at least 60%, at least 70%, at least 75%, at least 80%, at least 85%, at least 90%, at least 95%, at least 98%, at least 99%, at least 99.5% amino acid sequence identity to SEQ ID NOS: 10, 11, 12, or 13.

Another aspect of this disclosure provides a polynucleotide sequence encoding the recombinant fusion protein.

Still another aspect of this disclosure provides a recombinant expression vector including the polynucleotide sequence.

In an embodiment, the recombinant expression vector has at least 50%, at least 60%, at least 70%, at least 75%, at least 80%, at least 85%, at least 90%, at least 95%, at least 98%, at least 99%, at least 99.5% polynucleotide sequence identity to SEQ ID NOS: 22, 23, 24, or 25.

In an embodiment, the recombinant expression vector has a polynucleotide sequence of SEQ ID NOS: 22, 23, 24, or 25.

Still another aspect of this disclosure provides a recombinant expression vector including the polynucleotide sequence.

In an embodiment, the recombinant expression vector having the nucleic acid sequence encoding for at least one recombinant fusion protein may be inserted in a host cell and recombined with the host cell genome or refers to any nucleic acid including a nucleotide sequence competent to replicate spontaneously as an episome. Such a vector may include a linear nucleic acid, a plasmid, a phagemid, a cosmid, an RNA vector, a viral vector, etc.

In an embodiment, the vector may be genetically engineered to incorporate the nucleic acid sequence encoding the recombinant fusion protein in an orientation either N-terminal and/or C-terminal to a nucleic acid sequence encoding a peptide, a polypeptide, a protein domain, or a full-length protein of interest, and in the correct reading frame so that the recombinant fusion protein including NGF and SP or NGF and CGRP may be expressed. Expression vectors may be selected from those readily available for use in prokaryotic or eukaryotic expression systems.

Standard recombinant nucleic acid methods may be used to express a genetically engineered recombinant fusion protein. The nucleic acid sequence encoding the recombinant fusion protein according to one embodiment disclosed in the present application may be cloned into a nucleic acid expression vector, e.g., with appropriate signal and processing sequences and regulatory sequences for transcription and translation, and the protein may be synthesized using automated organic synthetic methods.

In order to obtain high level expression of a cloned gene or nucleic acid, for example, a cDNA encoding the recombinant fusion protein according to one embodiment disclosed in the present application, the recombinant fusion protein sequence may be typically subcloned into an expression vector that includes a strong promoter for directing transcription, a transcription/translation terminator, and in the case of a nucleic acid encoding a protein, a ribosome binding site for translational initiation. Suitable bacterial promoters are well known in the art. Bacterial expression systems for expression of the recombinant fusion protein are available in, e.g., E. coli, Bacillus sp., and Salmonella. Kits for such expression systems are commercially available. Eukaryotic expression systems for mammalian cells, yeast, and insect cells are well known in the art and are also commercially available. The eukaryotic expression vector may be preferably an adenoviral vector, an adeno-associated vector, or a retroviral vector.

The polynucleotide sequence according to one embodiment disclosed in the present application may be present in a vector in which the polynucleotide sequence is operably linked to regulatory sequences capable of providing for the expression of the polynucleotide sequence by a suitable host cell.

Within an expression vector, the term “operably linked” is intended to mean that the polynucleotide sequence of interest is linked to the regulatory sequence(s) in a manner which allows for expression of the polynucleotide sequence. The term “regulatory sequence” is intended to include promoters, enhancers, and other expression control elements. Such operable linkage with the expression vector can be achieved by conventional gene recombination techniques known in the art, while site-directed DNA cleavage and linkage are carried out by using conventional enzymes known in the art.

The expression vectors may contain a signal sequence or a leader sequence for membrane targeting or secretion, as well as regulatory sequences such as a promoter, an operator, an initiation codon, a termination codon, a polyadenylation signal, an enhancer and the like. The promoter may be a constitutive or an inducible promoter. Further, the expression vector may include one or more selectable marker genes for selecting the host cell containing the expression vector and may further include a polynucleotide sequence that enables the vector to replicate in the host cell in question.

The expression vector constructed according to an embodiment may be the vector where the polynucleotide encoding the recombinant fusion protein is inserted within the multiple cloning sites (MCS) of a pT7 vector.

The recombinant fusion protein may be introduced into an appropriate host cell, e.g., a bacterial cell, a yeast cell, an insect cell, or a tissue culture cell. The recombinant protein may also be introduced into embryonic stem cells in order to generate a transgenic organism. Large numbers of suitable vectors and promoters are known to those skilled in the art and are commercially available for generating the recombinant protein.

Known methods may be used to construct vectors including the polynucleotide sequence according to one embodiment disclosed in the present application and appropriate transcriptional/translational control signals. These methods include in vitro recombinant DNA techniques, synthetic techniques, and in vivo recombination/genetic recombination.

Another aspect of the embodiment provides a transformant transformed with the recombinant expression vector.

The transformation includes transfection and refers to a process whereby a foreign (extracellular) DNA, with or without an accompanying material, enters into a host cell. The “transfected cell” refers to a cell into which the foreign DNA is introduced into the cell, and thus the cell harbors the foreign DNA. The DNA may be introduced into the cell so that a nucleic acid thereof may be integrated into the chromosome or replicable as an extrachromosomal element. The cell with the replicable foreign DNA is called a transformant.

As used herein, “introducing” of a protein, a peptide, an organic compound into a cell may be used interchangeably with the expression of “carrying,” “penetrating,” “transporting,” “delivering,” “permeating” or “passing.”

It is understood that the host cell refers to a eukaryotic or prokaryotic cell into which one or more DNAs or vectors are introduced and refers not only to the particular subject cell but also to the progeny or potential progeny thereof. Because certain modifications may occur in succeeding generations due to either mutation or environmental influences, such progeny may not, in fact, be identical to the parent cell, but are still included within the scope of the term as used herein.

The host cells may be preferably bacterial cells, and as the bacterial cells, there are, in principle, no limitations. They may be eubacteria (gram-positive or gram-negative) or archaebacteria, as long as they allow genetic manipulation for insertion of a gene of interest, preferably for site-specific integration, and they may be cultured on a manufacturing scale. Preferably, the host cells may have the property to allow cultivation to high cell densities.

Examples of bacterial host cells that may be used in the preparation of the recombinant fusion protein are E. coli, Bacillus subtilis, Pseudomonas fluorescens as well as various Corynebacterium and Lactococcus lactis strains. Preferably, the host cells are Escherichia coli cells.

In an embodiment, the host cell may include an RNA polymerase capable of binding to a promoter regulating the gene of interest. The RNA polymerase may be endogenous or exogenous to the host cell.

In an embodiment, host cells with a foreign strong RNA polymerase may be used. For example, Escherichia coli strains engineered to carry a foreign RNA polymerase (e.g., like in the case of using a T7 promoter a T7-like RNA polymerase in the so-called “T7 strains”) integrated in their genome may be used. Examples of T7 strains, e.g., BL21 (DE3), HMS174 (DE3), and their derivatives or relatives (see Novagen, pET System manual, 11th edition), may be widely used and commercially available. Preferably, BL21-CodonPlus (DE3)-RIL or BL21-CodonPlus (DE3)-RIPL may be used. These strains are DE3 lysogens containing the T7 RNA polymerase gene under control of the lacUV5 promoter. Induction with IPTG allows production of T7 RNA polymerase which then directs the expression of the gene of interest under the control of the T7 promoter.

The host cell strains, E. coli BL21 (DE3) or HMS174 (DE3), which have received their genome-based T7 RNA polymerase via the phage DE3, are lysogenic. It is preferred that the T7 RNA polymerase contained in the host cell has been integrated by a method which avoids, or preferably excludes, the insertion of residual phage sequences in the host cell genome since lysogenic strains have the disadvantage to potentially exhibit lytic properties, leading to undesirable phage release and cell lysis.

The method for preparing the recombinant fusion protein includes preparing the recombinant expression vector: preparing the transformant using the recombinant expression vector; culturing the transformant; and recovering the recombinant fusion protein expressed by culturing.

Cultures may be preferably done in the presence a feed medium, in a the fed-batch mode, semi-continuous mode, or continuous mode. The bacterial expression host cells may include a DNA construct encoding the protein of interest under the control of a promoter that enables expression of said protein, integrated in their genome or not.

There are no limitations in the type of the culture medium. The culture medium may be semi-defined, i.e., containing complex media compounds (e.g. yeast extract, soy peptone, casamino acids), or it may be chemically defined, without any complex compounds. Preferably, a defined medium may be used. The defined media (also called minimal or synthetic media) are exclusively composed of chemically defined substances, i.e., carbon sources such as glucose or glycerol, salts, vitamins, and, in view of a possible strain auxotrophy, specific amino acids or other substances such as thiamine. Most preferably, glucose may be used as a carbon source. Usually, the carbon source of the feed medium serves as the growth-limiting component which controls the specific growth rate.

Host cells may be disrupted by any convenient method, including freeze-thaw cycling, sonication, mechanical disruption, or the use of cell lysing agents. There are a number of general methods known in the art for purifying recombinant (and non-recombinant) proteins. The methods may include, e.g., ion-exchange chromatography, size-exclusion chromatography, affinity chromatography, selective precipitation, dialysis, and hydrophobic interaction chromatography. These methods may be adapted to devise a purification strategy for the cell permeable recombinant fusion protein. If the cell permeable recombinant fusion protein includes a purification handle, such as an epitope tag or a metal chelating sequence, affinity chromatography may be used to easily purify the protein.

The amount of the protein produced may be evaluated by detecting the advanced macromolecule transduction domain directly (e.g., using Western analysis) or indirectly (e.g., by assaying materials derived from the cells for specific DNA binding activity, such as by electrophoretic mobility shift assay). Proteins may be detected prior to purification, during any stage of purification, or after purification. In some implementations, purification or complete purification may not be necessary.

In an embodiment, a pharmaceutical composition includes the recombinant fusion protein as an active ingredient.

The pharmaceutical composition is used for treating pain caused by osteoarthritis in a subject. The pharmaceutical composition includes an immunogenic recombinant fusion protein and a pharmaceutically acceptable formulation.

In an embodiment, the pharmaceutical composition may be for injectable (e.g. subcutaneous, intraperitoneal, intramuscular) and may include the active ingredient in an amount of 0.001 mg/kg to 1000 mg/kg, preferably 0.01 mg/kg to 100 mg/kg, more preferably 0.1 mg/kg to 20 mg/kg for human, more preferably 0.001 mg/kg to 10 mg/kg for dogs, more preferably 0.001 mg/kg to 5 mg/kg for cats, and more preferably 0.001 mg/kg to 20 mg/kg for horses.

For example, dosages per day normally fall within the range of about 0.001 to about 1000 mg/kg of body weight. In the treatment of adult humans, the range of about 0.1 to about 50 mg/kg/day, in single or divided dose, is especially preferred. However, it will be understood that the concentration of the recombinant fusion protein actually administered will be determined by a physician or veterinarian, in the light of the relevant circumstances, including the condition to be treated, the chosen route of administration, the age, weight, and response of the individual subject, and the severity of the patient's symptoms, and therefore the above dosage ranges are not intended to limit the scope of the invention in any way. In some instances, dosage levels below the lower limit of the aforesaid range may be more than adequate, while in other cases still larger doses may be employed without causing any harmful side effects, provided that such larger doses are first divided into several smaller doses for administration throughout the day.

The pharmaceutical composition according to an embodiment of this disclosure may be prepared by using pharmaceutically suitable and physiologically acceptable additives, in addition to the active ingredient, and the additives may include excipients, disintegrants, sweeteners, binders, coating agents, blowing agents, lubricants, glidants, flavoring agents, etc.

For administration, the pharmaceutical composition may be preferably formulated by further including one or more pharmaceutically acceptable carriers in addition to the above-described active ingredient.
For formulation of the composition into a liquid preparation, a pharmaceutically acceptable carrier which is sterile and biocompatible may be used, such as saline, sterile water, a Ringer's solution, buffered saline, an albumin infusion solution, a dextrose solution, a maltodextrin solution, glycerol, and ethanol, and these materials may be used alone or in any combination thereof. If necessary, other common additives, such as antioxidants, buffers, bacteriostatic agents, etc., may be added. Further, diluents, dispersants, surfactants, binders, and lubricants may be additionally added to prepare injectable formulations such as aqueous solutions, suspensions, and emulsions. Furthermore, the composition may be preferably formulated, depending upon diseases and ingredients, using any appropriate method known in the art.

In an embodiment, the recombinant fusion protein is use as a medicament for the treating pain caused by osteoarthritis in a subject.

In an embodiment, the medicament including at least one recombinant fusion protein that is capable of eliciting the production of antibodies.

In an embodiment, the recombinant fusion protein is used in the preparation of a medicament for the treating pain caused by osteoarthritis in a subject.

In an embodiment, the method of treating pain caused by osteoarthritis in a subject includes administering to the subject a therapeutically effective amount of the recombinant fusion protein used as an immunogen.

In an embodiment, the subject is a mammal.

In an embodiment, the subject is a dog, cat, horse, human or another mammal (e.g., mouse, rat, rabbit, cattle, swine, sheep, or primate) that can be afflicted with or is susceptible to osteoarthritis but may or may not have osteoarthritis.

In an embodiment, the amount effective or effective amount is the amount of an active ingredient or a pharmaceutical composition disclosed herein that when administered to a subject for treating a disease, is sufficient to effect such treatment of the disease. Any improvement in the subject is considered sufficient to achieve treatment. An effective amount of an active ingredient or a pharmaceutical composition disclosed herein, used for the treatment of osteoarthritic pain may vary depending upon the manner of administration, the age, body weight, and general health of the subject. Ultimately, the prescribers or researchers will decide the appropriate amount and dosage regimen.

In the method of treatment according to an embodiment of this disclosure, the immunogenic composition including the recombinant fusion protein as an active ingredient may be administered by intravenous, intra-arterial, intraperitoneal, intramuscular, intrasternal, percutaneous, topical, intraocular or subcutaneous route.

In an embodiment, the immunogenic composition is capable of simultaneously inhibiting in vivo the biological activity of two or more endogenous nociceptive molecules that act in concert to mediate pain signaling or perception, in the preparation of a medicament.

In an embodiment, the use of an immune composition includes an effective amount of an immunogenic compound which is capable of inducing a simultaneous immune response against two or more endogenous nociceptive molecules resulting in a therapeutic alleviation of a painful condition or disease.

In an embodiment, the immunogenic composition includes appropriate carriers and/or adjuvants to the immunogenic recombinant fusion protein to facilitate a simultaneous immune response against two or more nociceptive molecules that act in concert to mediate pain signaling or perception, in the preparation of a medicament or vaccine for the treatment of pain conditions.

In an embodiment, the NGF, SP and CGRP fragments may be modified by amino acid substitution, deletion and/or addition while retaining their immunogenicity Preferentially, the modifications in their amino acid sequences enhance their immunogenicity.

In an embodiment, the recombinant fusion proteins are expressed in bacteria and purified as inclusion bodies, thus enhancing the immunogenicity of the NGF, SP or CGRP fragments as compared to the native fragments.

In an embodiment, the specific amino acid sequences include in the recombinant fusion proteins are species-specific to the mammal that needs to be treated. The mammal includes canine, feline, equine and human.

In an embodiment, the immunogenic compounds are recombinant fusion peptides or polypeptides including at least 6 consecutive amino acids of one any of the sequences disclosed herein.

TABLE 1
Recombinant fusion proteins and immunogenic fragments

| | SEQ ID NO |
|---|---|
| Name | (amino acid) |
| proNGF Canis lupus | SEQ ID NO: 1 |
| proNGF Felis catus | SEQ ID NO: 2 |
| proNGF Equus caballus | SEQ ID NO: 3 |
| proNGF Homo sapiens | SEQ ID NO: 4 |
| NGF Canis lupus | SEQ ID NO: 5 |
| NGF Felis catus | SEQ ID NO: 6 |
| NGF Equus caballus | SEQ ID NO: 7 |
| NGF Homo sapiens | SEQ ID NO: 8 |
| Substance P | SEQ ID NO: 9 |
| CGRP Canis lupus | SEQ ID NO: 10 |
| CGRP Felis catus | SEQ ID NO: 11 |
| CGRP Equus caballus | SEQ ID NO: 12 |
| CGRP Homo sapiens | SEQ ID NO: 13 |

In an embodiment, the immunogenic fragment can include at least 6 consecutive amino acids of any of SEQ ID NOS: 1-13. (Table 1)

The immunogenic fragment of SEQ ID NO: 1 or SEQ ID NO: 5 can include one of the following sequences: SSSHPVFHRGEFS (SEQ ID NO: 14), DSVSVW (SEQ ID NO: 15), TDIKGK (SEQ ID NO: 16), YFFETKCR (SEQ ID NO: 17), YCTTTHTF (SEQ ID NO: 18), RIDTACVCV (SEQ ID NO: 19), and RGIDSKH (SEQ ID NO: 20). These peptides contain relevant sequences for the interaction of NGF with its TrkA and p75 receptors. Sequences 15 to 20 are conserved for the four species of interest.

The immunogenic fragment of SEQ ID NO: 4 or SEQ ID NO: 8 can include the following sequence: SSSHPIFHRGEFS (SEQ ID NO: 21). This peptide contains a specific modification at position 6 with respect to SEQ ID NO: 14 and corresponds to the human sequence of NGF. The recombinant fusion protein includes the combination of any of SEQ ID NOS: 1-21.

The recombinant fusion protein has at least 50%, at least 60%, at least 70%, at least 75%, at least 80%, at least 85%, at least 90%, at least 95%, at least 98%, at least 99%, at least 99.5% amino acid sequence identity with the aforementioned immunogenic fragments. The fragment retains the same immunological properties as the native peptide from which it derives, including the production of antibodies.

In an embodiment, the pain conditions are associated with OA pain, neuropathic pain, or cancer pain.

In an embodiment, a dog affected by a pain condition is treated with an immunogenic composition including at least one recombinant fusion protein comprising at least one immunogenic fragment derived from SEQ ID NO: 1 or SEQ ID NO: 5 and at least one immunogenic fragment derived from SEQ ID NO:9 or SEQ ID NO: 10.

In an embodiment, a cat affected by a pain condition is treated with an immunogenic composition including at least one recombinant fusion protein comprising at least one immunogenic a fragment derived from SEQ ID NO: 2 or SEQ ID NO: 6 and at least one immunogenic fragment derived from SEQ ID NO:9 or SEQ ID NO: 11.

In an embodiment, a horse affected by a pain condition is treated with an immunogenic composition including at least one recombinant fusion protein comprising at least one immunogenic fragment derived from SEQ ID NO: 3 or SEQ ID NO: 7 and at least one immunogenic fragment derived from SEQ ID NO:9 or SEQ ID NO: 12.

In an embodiment, a human affected by a pain condition is treated with an immunogenic composition including at least one recombinant fusion protein comprising at least one immunogenic fragment derived from SEQ ID NO: 4 or SEQ ID NO: 8 and at least one immunogenic fragment derived from SEQ ID NO:9 or SEQ ID NO: 13.

In an embodiment, the immunogenic composition includes an effective amount between 0.5 ug and 10000 μg of said recombinant fusion protein.

In an embodiment, the immunogenic composition includes urea as an adjuvant.

In an embodiment, the adjuvant includes an oil-in-water adjuvant, a polymer and water adjuvant, a water-in-oil adjuvant, an aluminum hydroxide adjuvant or combinations thereof.

In an embodiment, the recombinant fusion proteins are produced as inclusion bodies which are solubilized in urea 4-8M.

In an embodiment, the immunogenic composition includes a plurality of recombinant fusion protein capable of inducing an immune response against at least two different nociceptive mediators.

In an embodiment, the immunogenic composition comprises a combination or simultaneous co-administration of monoclonal antibodies against two or more nociceptive mediators instead of the active immunization.

In an embodiment, the immunogenic composition is used in an active immunization protocol.

In an embodiment, the administration of the immunogenic composition reduces pain signs, perceived pain scores by the companion animal owners or need of consumption of pain medications, as compared with pain before said administration.

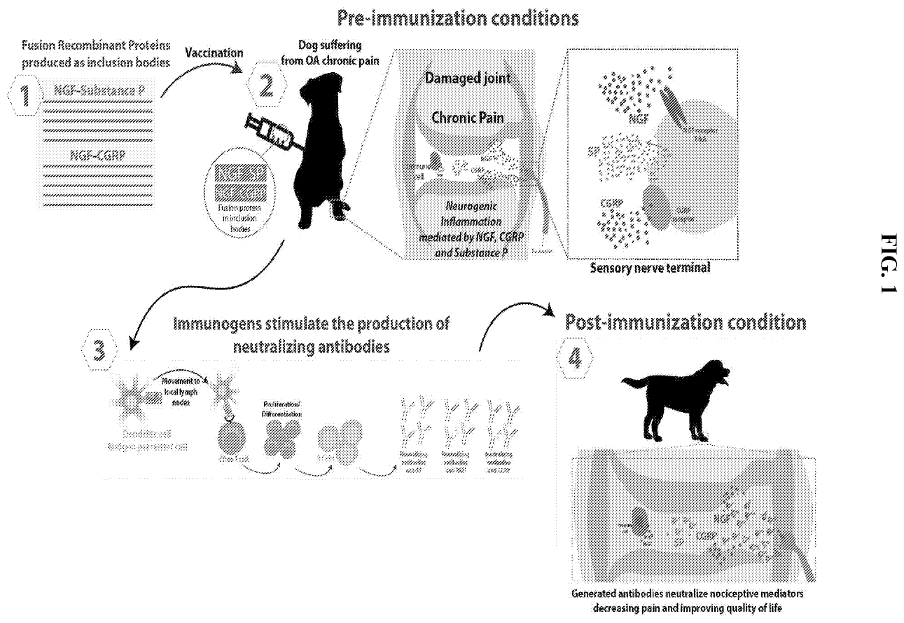

FIG. 1 shows an embodiment of the design, production and

immunization with the recombinant fusion NGF-SP immunogen in osteoarthritic dogs suffering from chronic refractory pain. In the first step, fusion recombinant fusion proteins containing NGF, SP and/or CGRP DNA sequences can be expressed via an expression vector in bacteria such as Escherichia Coli which may be in the form of inclusion bodies. Fusion immunogens can be purified, and bacterial DNA, protein and endotoxins can be removed before vaccine formulation in Urea and adjuvant (Montanide™ Gel 01, SEPPIC). In the second step, NGF-SP immunogen can be subcutaneously administered to client-owned dogs living with osteoarthritis that show clinical signs of chronic pain that perturb significantly their normal day-to-day life. All dogs included in the clinical trials showed refractory OA to other pharmacological treatments, that were not removed before starting the present protocol. The immunization protocol includes a priming followed by three boosters every 15 days using the same formulation and concentration (5 mg). In the third step, serum antibody titers can be measured before starting the immunization protocol, before boosters and 15 and 90 days after the last booster. Significant antibody titers of IgG against NGF and Substance-P can be detected in the serum of all immunized dogs. Also, neutralizing activity of IgG can be analyzed in cellular cultures. Neutralizing activity against NGF and SP can be demonstrated in culture. In the fourth step, active immunization using NGF-SP immunogen significantly reduces chronic pain in dogs but also improves dogs' function affected by pain. Pain medication consumed by all dogs are significantly reduced after the immunization protocol, and no dogs remain on corticoids or opioids. No significant adverse events are recorded and all dogs show signs of significant improvement up to 90 days after completion of immunization protocol.

### EXAMPLES

Specific embodiments will now be demonstrated by reference to the following examples. It should be understood that these examples are disclosed solely by way of illustrating the invention and should not be taken in any way to limit the scope of the present invention.

Active immunization with a recombinant fusion protein NGF-SP or NFP-CGRP reduces OA-associated pain in dogs

16 adult dogs were included in a non-controlled, open-labeled study. Inclusion criteria include chronic osteoarthritis pain condition causing profound disability that was unresponsive to current anti-inflammatory treatments, thus being considered for euthanasia. Dogs were submitted to systemic immunization using a recombinant fusion protein bearing antigenic sites for NGF and SP as an add-on therapy to previous treatments or interventions. The vaccine was administered subcutaneously using 5 mg of the NGF-SP immunogen every 2 weeks for a total of 4 injections. Serum antibody levels to NGF and SP were recorded throughout the study and correlated with clinical responses. Pain and general activity and function were evaluated throughout the immunization period and continued for 3 months after the last immunization in each dog with CBPI, compiled by the dog owners, as well as a slightly modified Colorado State canine acute pain scale (scoring 0 for no pain, to 4 in case of severe pain or lack of function), used by the clinician. Pain medications used in dogs were also recorded throughout the study period.

All patients underwent the 4-injection scheme. Occasional local inflammation and short-lived systemic effects were observed following injections. Titers of IgG to NGF and Substance P systemically increased to 1:8,000-30,000 and 1:6,000-64,000, respectively, by 2 weeks after completion of the immunization scheme. Compared with initial baseline clinical scores (pain on palpation mean score: 3.7: function/lameness mean score: 3.6: general activity mean score: 3.2), the total veterinarian scores were significantly lower (pain on palpation mean score: 1.8-1.7; function/lameness mean score: 2.1-1.8: general activity mean score: 2.2-1.9) at weeks 2 and 20, respectively, after the last immunization. Similarly, CBPI owner assessments showed significant improvements from baseline after 2 weeks from the last immunization and continuing until 20 weeks (p<0.05). All dogs also showed a drastically decreased consumption of pain drugs, including NSAIDs and corticosteroids.

In this open, non-controlled study evaluating combined NGF-SP vaccination in 16 client-owned dogs, veterinarian and owners' assessments showed statistically significant reductions in OA pain, need for pain medication and increase functional activity s. The immunizations were well tolerated and no dog reached an end-stage disease or required euthanasia. These results show promise for the management of canine OA.

### Example 1: DNA Sequences (Canis lupus familiaris)

In an embodiment, plasmid pT7 was used as an expression vector to clone the recombinant DNA. The synthesis of the genes and their cloning in the pT7 vector was carried out by Genscript (Project U595MEB120). All sequences were optimized for their expression in E. coli and inserted into T7 plasmid between BamHI and XhoI restriction sites. Similar methodologies can be implemented to prepare recombinant DNAs of interest for felines, equines, and humans coding for NGF or Pro NGF, and SP or CGFR peptides set forth in SEQ ID NOS: 2-4, 6-9, and 11-13, and their functional equivalents for the respective species.

In an embodiment, nucleic acid sequences of certain recombinant pT7 plasmids are SEQ ID NOS: 22-25.

TABLE 2
Recombinant vector having a nucleic acid sequence
encoding a recombinant fusion protein

| | SEQ ID NO: |
|---|---|
| Name | (nucleic acid) |
| pT7 proNGF-SP | SEQ ID NO: 22 |
| pT7 NGF-SP | SEQ ID NO: 23 |
| pT7 Chimera NGF-SP | SEQ ID NO: 24 |
| pT7 NGF-CGRP | SEQ ID NO: 25 |

### Example 2: Recombinant Fusion Protein (Canis lupus familiaris)

As examples, the amino acid sequences of certain recombinant fusion protein are SEQ ID NOS: 26-29. Similar methodologies can be implemented to prepare recombinant fusion protein for felines, equines, and humans incorporating NGF or ProNGF, and SP or CGFR peptides set forth in SEQ ID NOS: 2-4, 6-9, and 11-13, and their functional equivalents for the respective species. (Table 1.)

TABLE 3
Recombinant fusion proteins

| Recombinant | |
|---|---|
| fusion protein | SEQ ID NO |
| ProNGF-SP | SEQ ID NO: 26 |
| NGF-SP | SEQ ID NO: 27 |
| Chimera NGF-SP | SEQ ID NO: 28 |
| NGF-CGRP | SEQ ID NO: 29 |

### Example 3: Expression and purification of NGF-SP on inclusion

bodies

To express the fusion recombinant immunogens proNGF-SP, NGF-SP, NGF-CGRP and a chimera containing NGF-SP synthetic genes, the plasmid pT7 with recombinant genes were transformed into BL21 (DE3) chemically competent E. coli (EC0114, Thermo Fischer), according to the manufacturer's instructions. Transformed E. coli cells are grown at 37° C. in Lauria-Bertani Broth containing 100 μg/mL Ampicilin (SIGMA A0166) overnight at 37° C. and 220 rpm. Then one liter of Terrific Broth was inoculated with 10 mL of pre-culture and incubated at 37° C. and 220 rpm up to an optical density at 600 nm of 2 followed by induction with 1 mM IPTG (EUROMEDEX EU0008-B) for 4 hours at 37° C. and 220 rpm. The cells were harvested 10 minutes at 10000×g and 4° C. after 4 hours and resuspended in buffer 30 mM Tris pH8, 150 mM NaCl and 0.5 mg/mL lysozyme. Cell suspension was sonicated (1:40 minutes: 20 seconds ON and 1 minute OFF at 50%) and centrifuged 40 minutes at 20000×g and 4° C. The pellet was resuspended in Washing Buffer I (WBI, 50 mM Tris pH8, 50 mM NaCl, 0.5% Triton X-100, 1.5 mM B-Mercaptoethanol, 1.6M Urea) and centrifuged 20 minutes at 20000×g and 4° C., this step is repeated. The pellet was resuspended in Washing Buffer II (WBII, 30 mM Tris pH8, 150 mM NaCl) and centrifuged 20 minutes at 20000×g and 4° C., this step was repeated two times. The pellet was resuspended in 20 mM Tris pH8, 500 mM NaCl, 8M Urea. The protein then was purified by IMAC. All chromatography experiments are performed at room temperature. The protein in 8M Urea is applied slowly with a maximum of 2 mL/min flow rate onto a pre-equilibrated (with protein buffer) 5 mL HisTrap chelating HP column charged with Ni+2 (GE17-5248-02, Cytiva). The column is then washed with 5 column volumes (CV) of protein buffer. The protein is eluted in 3 steps of 10 mL with elution buffer (20 mM Tris pH 8, 500 mM NaCl, 500 mM Imidazole, 8M Urea). All fractions are analyzed by SDS-PAGE and dialyzed to remove imidazole.

Similar methodologies can be implemented to prepare inclusion bodies of interest for felines, equines, and humans.

### Example 4: Removal of Endotoxins by Triton X-114

The pellet obtained in last step of Example 3 was resuspended in WBII and 1% Triton X-114 by sonication (1:30 minutes: 15 seconds ON and 1 minute OFF at 45%) and incubated 30 minutes on ice, then 30 minutes at 50° C. and centrifuged at 20000×g 20 minutes at 20° C. This step was repeated 10 times. Final pellet was washed three times with WBII and resuspended in 20 mM Tris pH8, 500 mM NaCl, 8M Urea.

### Example 5: Removal of Endotoxins by Magnetic Beads

The pellet obtained in Example 4 was resuspended in WBII supplemented with 1% Triton X-114 and sonicated (1:30 min: 15 sec ON and 1 min OFF at 45%) and incubated on ice during 30 min, then incubated at 50° C. during 30 min and centrifuged 20 min at 20000 g at 20° C. This step was repeated 10 times. Final pellet was resuspended in 20 mM Tris pH8, 500 mM NaCl and 8 M Urea. To complement endotoxin and bacterial DNA removal, pellet was incubated with endotoxin removal beads (Mylteni, #130-093-657) and manufacturer instructions were followed. The level of endotoxins was measured by LAL Chromogenic Endotoxin Quantitation Kit (Pierce, #88282) following manufacturer instructions.

### Example 6: Endotoxin Measurement

The level of endotoxins was measured by Pierce™ Chromogenic Endotoxin Quant Kit (Thermo Fisher A39553) utilized according to the manufacturer's instructions.

### Example 7: Protein Quantification

The proteins were measured by Bradford and the standard curve was made in Tris 20 mM pH8, NaCl 500 mM, Urea 8M. The amount and purity of protein was confirmed by mass densitometry in Acrylamide gels and by 2D electrophoresis. The identity of the sample was confirmed by mass spectrometry.

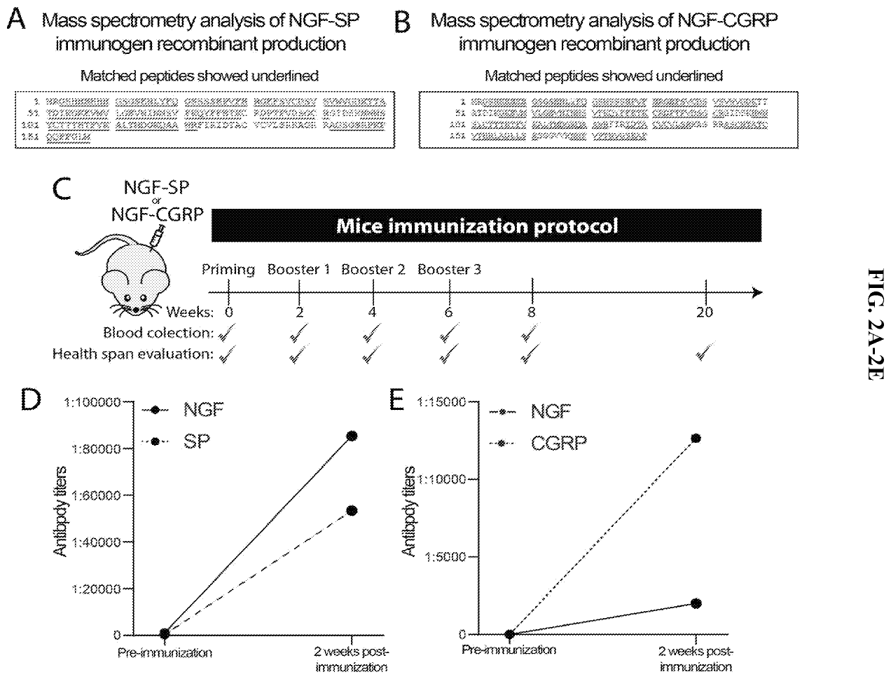

FIGS. 2A and 2B show mass spectrometry analysis of recombinant fusion immunogens, NGF-SP and NGF-CGRP, respectively. Matched peptides are shown underlined. There is a statistically significant recognition of NGF-SP and NGF-CGRP, respectively.

### Example 8: Safety, Tolerance and Immune Response in Mice

To evaluate the tolerance, safety and immunological response elicited by the divalent peptides, three mice were immunized with 5 mg/kg of total protein every 15 days (prime-boost immunization protocol, with 3 boosters). The first immunization was performed using Freund's complete adjuvant and the remaining three boosters using incomplete Freund's adjuvant. Animals were followed daily to determine potential side effects of the immunization. To determine the immunological response and antibody production, blood samples were taken before the first immunization and 7 days after each administration. Animals were followed two months after the last booster to observer potential side effects of the immunization protocol. All procedures using laboratory animals were performed under the national and international guidelines and were approved by the Institutional Animal Committee for animal experimentation (CEUA Approved protocol: #012-16 to Dr. Martina Crispo). This study was carried out in strict accordance with the Institut Pasteur de Montevideo Committee's requirements and under the current ethical regulations of the Uruguayan Law N° 18.611 for animal experimentation that follow the Guide for the Care and Use of Laboratory Animals of the National Institutes of Health (USA).

FIG. 2C shows that mice were immunized with a formulation containing 5 mg/Kg of immunogens and complete Freund's adjuvant and 4M Urea in the priming and incomplete adjuvant in the following 3 boosters every 15 days. Blood samples were taken before the priming, before every booster and 2 weeks after the completion of the immunization protocol. Mice health span was evaluated daily by two trained operators up to 12 weeks after the completion of the immunization protocol. No significant adverse events were registered during the entire process.

FIGS. 2D and 2E show that immunization with the NGF-SP and NGF-CGRP recombinant fusion proteins, respectively, was safe, did not generate side effects and triggers an immune response in mice after 4 doses, with significant production of antibody titers against NGF, SP and CGRP.

The results show that that recombinant fusion protein coding for NGF-SP and NGF-CGRP used as immunogen elicit anti-NGF, anti-SP and anti-CGRP antibodies upon active immunization in mice.

### Example 9: Vaccine Formulation

Each dose was prepared with 20 mM Tris pH8, 500 mM NaCl, 4M Urea, 1-20% of Montanide™ Gel 01 PR (SEPPIC) and 2.5, 5 or 10 mg of inclusion bodies, depending on the dog's weight.

Similar methodologies can be implemented to vaccines of interest for felines, equines, and humans.

### Example 10: Safety, Tolerance and Immune Response in Horses

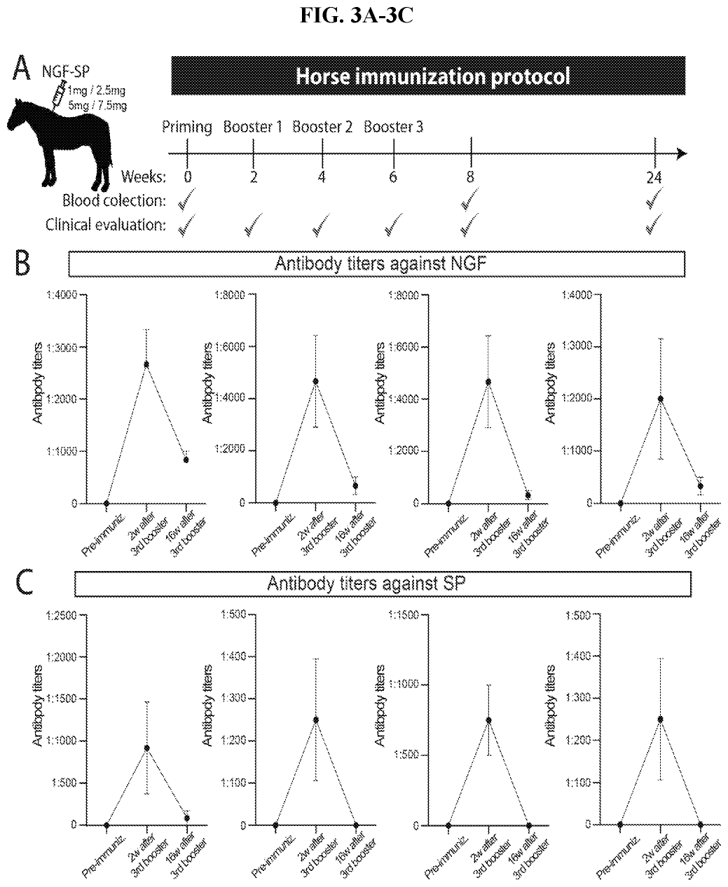

All procedures using healthy companion owned horses were performed under the national and international guidelines and were approved by the Institutional Animal Committee for animal experimentation (CEUA Approved protocol: #017-19 Annex II to Dr. Luis Barbeito). This study was carried out in strict accordance with the Institut Pasteur de Montevideo Committee's requirements and under the current ethical regulations of the Uruguayan Law No 18.611 for animal experimentation that follow the Guide for the Care and Use of Laboratory Animals of the National Institutes of Health (USA). All procedures were supervised during the entire procedure by two experts in veterinary medicine. 12 client-owned healthy horses were recruited to the study in a horse stable located in Canelones, Uruguay. Horse owners received a detailed description of the protocol and signed an informed consent form before the screening process. Owners incurred in no costs for participation. After the screening of potential candidates, a total of 12 horses were recruited in the study. Systemic subcutaneous immunization using NGF-SP immunogen was started on day 0 and boost injections were repeated on days 15, 30, and 45 as described in FIG. 3A. Blood was collected before priming and each booster. Also, blood was collected 2 weeks and 12 weeks after completion of the immunization. Horses were randomly divided in 4 experimental groups, 1, 2.5, 5 and 7.5 mg of recombinant fusion protein suspended in 1 ml of 5% Montanide™ Gel 01 (Seppic) adjuvant containing 4M Urea.

### Example 11: Immunization Timeline, Serum Extraction, and Inclusion Criteria for Dogs Living with Severe Osteoarthritis

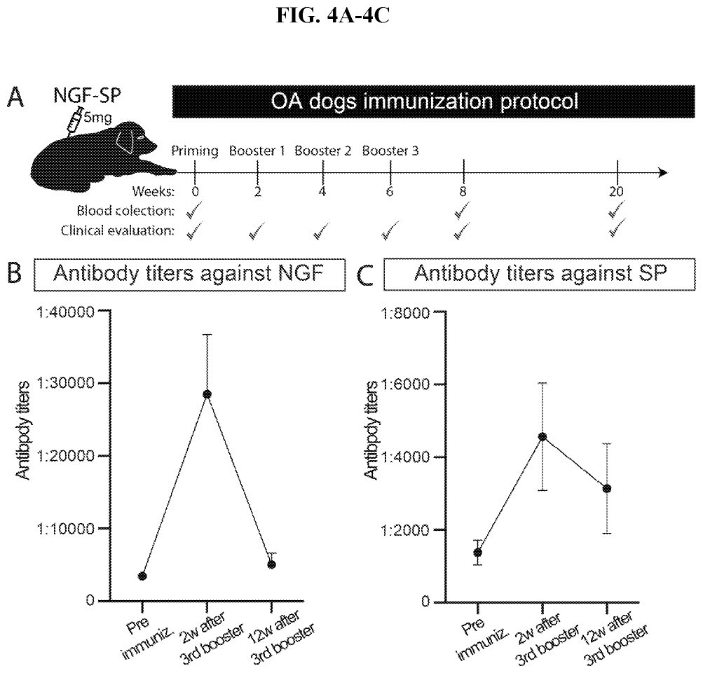

All procedures using companion owned dogs were performed under the national and international guidelines and were approved by the Institutional Animal Committee for animal experimentation (CEUA Approved protocol: #017-19 to Dr. Luis Barbeito). This study was carried out in strict accordance with the Institut Pasteur de Montevideo Committee's requirements and under the current ethical regulations of the Uruguayan Law N° 18.611 for animal experimentation that follow the Guide for the Care and Use of Laboratory Animals of the National Institutes of Health (USA). All procedures were carried on in the veterinary clinic of Dr. Gabriel Semiglia and supervised during the entire procedure by three experts in veterinary medicine. The study had an open label, add-on design using naturally occurring OA in dogs with profound disability that was unresponsive to current anti-inflammatory treatments, thus being considered for imminent euthanasia. 16 client-owned dogs with clinical OA related lameness despite the use of medications were recruited to the study in a veterinary clinic located in Montevideo, Uruguay. Pet owners received a detailed description of the protocol and signed an informed consent form before the screening process. Patients incurred in no costs for participation and were compensated with a long-term supply of treatment upon completion of the study if medically indicated. After the screening of potential candidates, a total of 16 dogs were recruited in the study. Systemic immunization using homogenate NGF-SP immunogen was started on day 0 and boost injections were repeated on days 14, 15, and 45 as shown in FIG. 4A. Blood was collected before priming and each booster. Also, blood was collected 2 weeks and 12 weeks after completion of the immunization. The vaccine composition and dosing were decided based on previous observations (data not shown). Dogs weighing 10-67 kg were administered with 5 mg of the recombinant fusion proteins, suspended in 1 ml of Seppic adjuvant containing 4M urea.

Domesticated dogs suffering from osteoarthritis were immunized as using NGF-SP immunogen as described in FIG. 4A. After the primer immunization, 3 boosters every two weeks were administered. Blood samples were taken before and after every immunization. Also, three months after the last booster a blood sample was taken to analyze antibody titers decline with time.

### Example 12: Inclusion Criteria

To be included in the study, it was decided a priori that eligible dogs (1-year-old or more and weight of 5 Kg or more) were required to (a) have lameness and owner-identified mobility impairment despite the use of medications lasting at least 3 months (b) exhibit a painful response upon manipulation of one or more joint that also may have radiographic changes consistent with chronic OA, (c) have turned refractory to current medications including a full dose of NSAIDs, corticoids or opioids, to ease pain and mobility impairment thus facing an indication of imminent euthanasia, d) be free from other clinically or laboratory detectable systemic disease. Dogs were excluded from the study if impending changes in the owner's ability to support dog health were expected during the study period.

### Example 13: Measurement of Serum Antibody Titers by ELISA in Dogs and Horses Systemically Immunized with NGF-SP Immunogen

The plate was coated with 1-2.5 μg/mL of NGF (CYT-579, Prospec) or 1-5 μg/mL of Substance P (RP10178, Genscript) in Carbonate Buffer 0.05M pH 9.6 overnight at 37° C. Plate was blocked with Milk 3% in PBS for 1 h at 37° C. After blocking, plate was incubated with serial dilutions of the serum in 3% Milk, PBS, 0.5% Tween 20 for 1 h at 37° C. Then, plates were washed with PBS-Tween-20 0.5% four times. The secondary antibodies (ab6789, ab102396 and ab112852) were incubated in the same conditions. After washing four times, the plates were revealed with TMB substrate solution (T0440, SIGMA) and the reaction was stopped with 1M H₂SO₄. Measurements was made in a Multiskan FC Microplate Photometer at 450 nm. Antibody titers are calculated as the highest dilution equal to or greater than 2.1* Blank.

Antibody titers were detected for both NGF and SP in healthy horses and dogs living with OA. In horses, significant antibody titers against NGF and SP were observed 2 weeks after the third booster (60 days from start) as shown in FIG. 3B and FIG. 3C. In dogs, the most significant peak in the antibody titers occurred two weeks after the last booster and decreased 90 days after the last booster, but they remained above the respective cut-off points even after 90 days after the last booster. FIG. 4B and FIG. 4C shows antibodies generated in dogs against NGF (B) and SP (C).

The results show that immunization with NGF-SP immunogen in dogs with osteoarthritis and healthy horses, elicited NGF and Substance P antibody titers.

### Example 14: Pain and Functional Assessment by Owners in Dogs Living with Advances Stages of OA

Clients completed a CBPI questionnaire addressing the dog's osteoarthritis pain and function before enrolment (Day −1) and at each follow-up visit thereafter (days 15±3, 30±3, 45±3 and 60±3). Day −1 served as the baseline score for the Scale. Questions 1-4 in the CBPI were summed to establish pain severity score (PSS) and questions 5-10 were summed to establish a pain interference score (PIS). Only dogs with a PSS and PIS≥2 were enrolled.

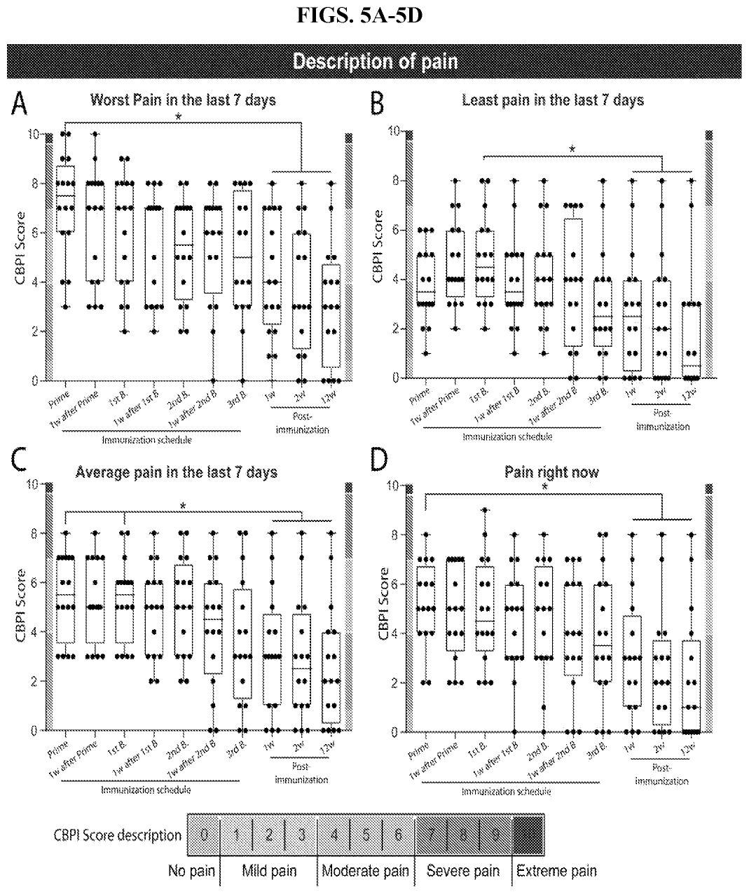

The dogs' owners CBPI scores were utilized to monitor signs and behaviors related to pain. As shown in FIGS. 5A-5D, all different parameters indicative of pain were significantly reduced by 1, 2 and 12 weeks after completion of the immunization period.

FIG. 5A shows that, according to dogs' owners, the mean worst pain in a 1-10 scale in the past week significantly decreases from 7.8 to 3 and to 2.9 after 2 weeks and after 12 weeks post-immunization, respectively. FIG. 5B shows that the mean least pain the past week significantly decreases from 3.5 to 2 and to 1 after 2 weeks and after 12 weeks post-immunization, respectively. FIG. 5C shows that the mean average pain during the past week significantly decreases from 5.4 to 2.8 and to 2.6 after 2 weeks and after 12 weeks post-immunization, respectively. FIG. 5D shows that the mean of the pain at the moment of veterinarian revision significantly decreases from 5.2 to 2.5 and to 2 after 2 weeks and 12 after weeks post-immunization, respectively. These results show that vaccination with NGF-SP immunogen in dogs with osteoarthritis significantly decreases chronic pain and that the pain continues to decrease post-immunization.

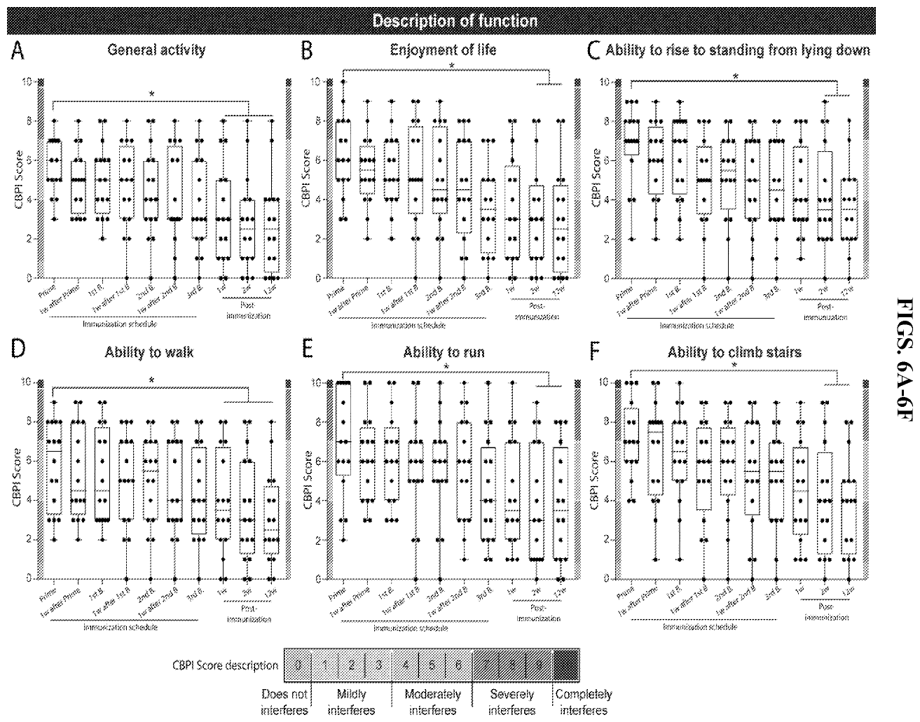

As shown in FIGS. 6A-6F, the CBPI scale filled by the dogs' owners during and after the immunization period showed a significant decrease in different functions 2 and 12 weeks after completing the immunization period. The scale describes how during the last 7 days pain has interfered with dogs' quality of life.

FIG. 6A shows that the general activity score significantly decreases from 5.8 to 2.6 after 12 weeks from the last booster. FIG. 6B shows that pain interference with the enjoyment of life significantly decreases from 6.2 to 2.8 after 12 weeks from the last booster. FIG. 6C shows that the ability of dogs to rise to stand from lying down significantly improves going from a score of 6.9 to 3.5 after 12 weeks from the last booster. FIGS. 6D and 6E show that the ability to walk and run significantly improves from 6.2 to 3 and from 7 to 3.6, respectively, after 12 weeks from the last booster. FIG. 6F shows that the pain interference with the ability to climb stairs significantly decreases from 7.1 to 3.8.

These results show that the active immunization NGF-SP immunogen in dogs with osteoarthritis significantly decreases chronic pain as assessed by CBPI.

### Example 15: Statistical Analysis

The Wilcoxon signed test was used to compare pain severity and pain interference scores before and after treatment. Univariate linear regression analysis was performed to determine factors (age, body weight, sex, breed, and baseline pain severity and pain interference scores) associated with the change in pain severity and pain interference scores. Two-tailed assessments were used for all analyses, and values of P<0.05 were considered significant.

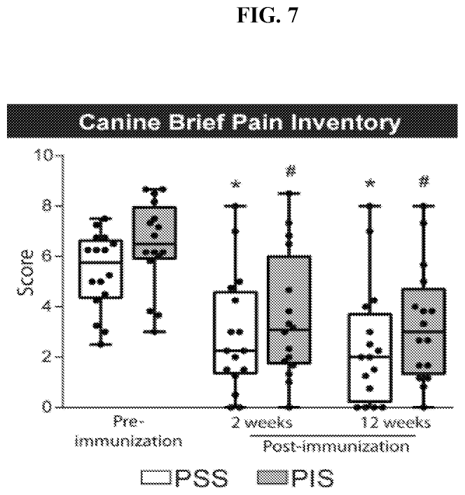

The CBPI is composed of pain severity score (PSS) and pain interference score (PIS). As shown in FIG. 7, after vaccination of NGF-SP, both PSS and PIS scores significantly decreased from 5.4 and 6.5 to 2.4 and 3.3, respectively, after 12 weeks from the last booster. The results show that the active immunization with NGF-SP significantly decreases the CBPI-based Pain Severity Score (PSS-CBPI) and the CBPI-based Pain Interference Score (PIS-CBPI).

### Example 16: Veterinarian Assessment of Lameness and Response to Palpation

The Colorado State University canine acute pain scale, with minor modifications, was used to assess lameness and pain by veterinarians. The secondary outcome measures were the clinical assessment of lameness, weight bearing, and reaction to joint manipulation made by a veterinarian and the record of medications needed to ease the dog's pain, mobility impairment and quality of life. Evaluations were performed by the investigators. No attempt was made to have the same investigator perform all assessments in any given dog.

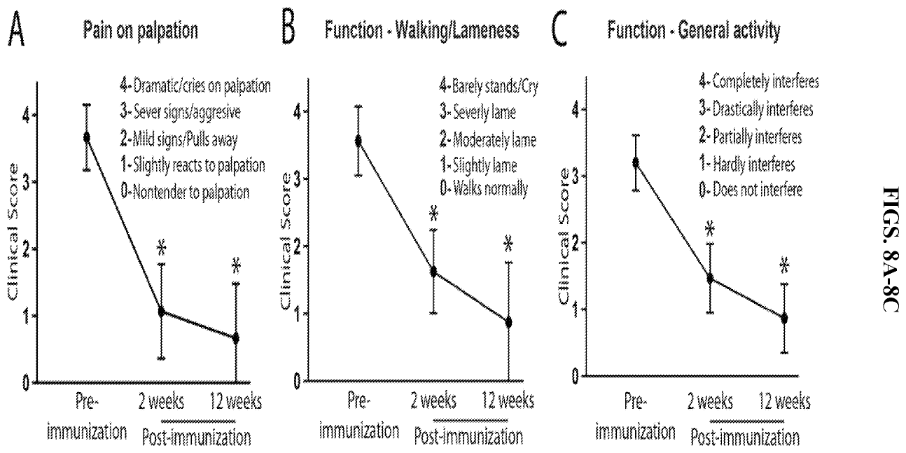

As shown in FIGS. 8A-8C, the Colorado State University canine acute pain scale was used to assess the clinical pain and lameness status of dogs, before and after vaccination. Parameters such as pain of palpation of affected joint, walking/lameness and general motor and behavioral activity were significantly decreased by 50% by 12 weeks after the immunization period.

FIG. 8A shows that the pain on palpation scores significantly decreased from 3.7 to 1.8 and to 1.6 after 2 weeks and after 12 weeks from the last booster. FIG. 8B shows that the dogs' function measured by walking capacity and lameness significantly improves after NGF-SP immunization, decreasing from 3.6 to 2.1 and to 1.9 after 2 weeks and after 12 weeks from the last booster. FIG. 8C shows that the interference of the pain with the dogs' general activity significantly decreased from 3.2 to 2.2 and to 1.8 after 2 weeks and 12 after weeks from the last booster.

The results show that the active immunization with the recombinant fusion protein NGF-SP significantly decreases dogs' pain and improves the quality of life as assessed by veterinarians.

### Example 17: Pain medication consumption

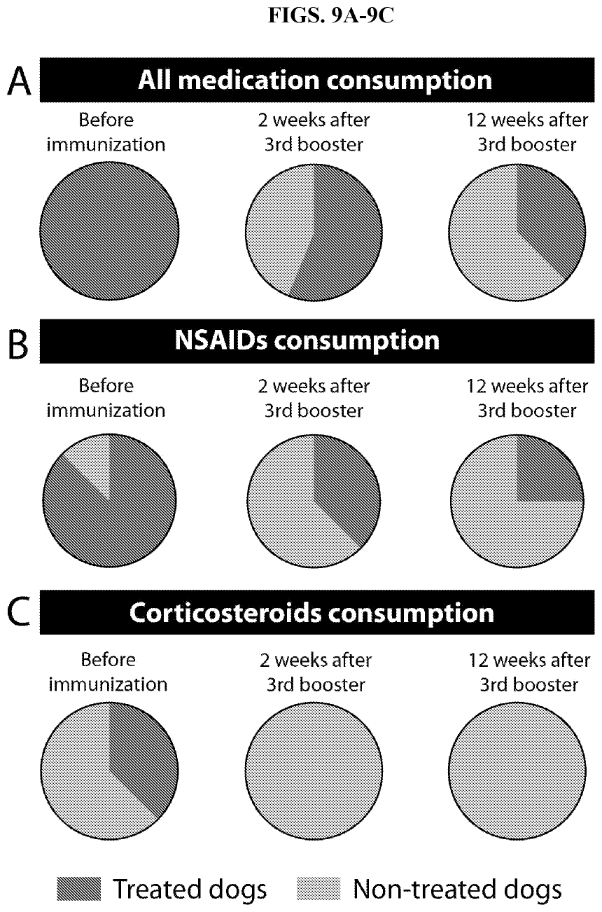

As shown in FIGS. 9A-9C, 100% of dogs received one or more pain medications before receiving the NGF-SP vaccine. After vaccination, the percentage of dogs receiving pain medications dropped to less than 50% and 30% by 2 and 20 weeks after the immunization period. Chronic corticoid treatments used in a subset of dogs presenting refractory OA were completely discontinued in vaccinated dogs.

FIG. 9A shows that before starting immunization, all dogs were treated with NSAIDs, corticosteroids, or opioids. 2 weeks after the last booster of immunization 55% of the immunized animals were free of medication. The follow up of the dogs shows that 12 weeks after the last booster, only 27% of the dogs still consume some kind of pain medication. FIG. 9B shows the analysis of NSAID consumption in dogs involved in the clinical trial. 12 weeks after finishing the immunization protocol, only 27% of the animals still consume some kind of NSAID, compared with 80% of the dogs that used to consume this kind of pain medication. FIG. 9C shows that before the clinical trial, 40% of the dogs needed some kind of corticosteroids. 2 weeks after the last booster, no dogs were consuming corticosteroids. The follow-up shows that no dogs returned to corticosteroid consumption 12 weeks after the last booster.

The results show that the active immunization with NGF-SP recombinant fusion protein decreased the consumption of pain medications in dogs with ostcoarthritis pain.

1. An immunogenic recombinant fusion protein comprising:
at least one immunogenic fragment derived from a nerve growth factor (NGF); and
at least one immunogenic fragment derived from substance P (SP) or calcitonin gene-related peptide (CGRP)
wherein the recombinant fusion protein is capable of eliciting a protective immune response against a nociceptive pain by eliciting the production of neutralizing antibodies.

2. The immunogenic recombinant fusion protein of claim 1, wherein the neutralizing antibodies are neutralizing antibodies against one or more of NGF, SP or CGRP.

3. (canceled)

4. The immunogenic recombinant fusion protein of claim 1, comprising an amino acid sequence having at least 90%, at least 95% or 100% sequence identity with SEQ ID NO: 26, SEQ ID NO: 27, SEQ ID NO: 28 or SEQ ID NO: 29.

5. The immunogenic recombinant fusion protein of claim 1, wherein the at least one immunogenic fragment derived from NGF comprises an amino acid sequence having at least 90%, at least 95% or 100% sequence identity with SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

6. (canceled)

7. The immunogenic recombinant fusion protein of claim 1, wherein the at least one immunogenic fragment comprises an amino acid sequence having at least 90%, at least 95% or 100% sequence identity with SEQ ID NO: 14, SEQ ID NO: 15, SEQ ID NO:16, SEQ ID NO: 17, SEQ ID NO:18, SEQ ID NO: 19, SEQ ID NO:20 or SEQ ID NO: 21.

8. (canceled)

9. (canceled)

10. (canceled)

11. The immunogenic recombinant fusion protein of claim 1, wherein the SP amino acid sequence comprises (a) at least one immunogenic fragment having at least 6 consecutive amino acids of SEQ ID NO: 9 or at least one immunogenic fragment having an amino acid sequence of at least 90%, at least 95% or 100% sequence identity with SEQ ID NO: 9.

12. The immunogenic recombinant fusion protein of claim 1, comprising an CGRP amino acid sequence having at least 90%, at least 95% or 100% sequence identity with SEQ ID NO: 10, SEQ ID NO: 11, SEQ ID NO: 12 or SEQ ID NO: 13.

13. (canceled)

14. The immunogenic recombinant fusion protein of claim 1, comprising SEQ ID NO: 27 or SEQ ID NO: 29.

15. (canceled)

16. A immunogenic recombinant vector comprising at least a nucleic acid encoding the recombinant fusion protein of claim 1.

17. The immunogenic recombinant vector of claim 16, comprising a nucleic acid sequence having at least 90%, at least 95% or at least 99% sequence identity with SEQ ID NO: 22, SEQ ID NO: 23, SEQ ID NO: 24 or SEQ ID NO: 25.

18. A method for producing the immunogenic recombinant fusion protein of claim 1, wherein said immunogenic recombinant fusion protein is produced in a procaryotic or eukaryotic expression system as an inclusion body.

19. (canceled)

20. The method of claim 18, wherein the immunogenic recombinant fusion protein is expressed from a recombinant vector.

21. (canceled)

22. An immunogenic composition comprising at least one immunogenic recombinant fusion protein of claim 1.

23. The immunogenic composition of claim 22, further comprising an acceptable carrier and/or an adjuvant selected from the group consisting of oil-in-water adjuvant, polymer and water adjuvant, water-in-oil adjuvant, aluminum hydroxide adjuvant and combinations thereof, wherein the adjuvant is preferably the complete or incomplete Freund's adjuvant, a Montanide™, aluminum salts (alum), oil emulsions, saponins, immune-stimulating complexes (ISCOMs), liposomes, microparticles, nonionic block copolymers, derivatized polysaccharides, cytokines, or bacterial derivatives.

24. (canceled)

25. (canceled)

26. (canceled)

27. An immunogenic vaccine for treating or preventing pain in a subject comprising at least one immunogenic recombinant fusion protein of claim 1 and a pharmaceutically acceptable carrier.

28. The immunogenic vaccine of claim 27, wherein the pain is associated with OA, neurogenic inflammation, neuropathy, rheumatoid arthritis, post-surgery or cancer.

29. The immunogenic vaccine of claim 27, wherein the pain is a one or more of nociceptive-related pain, inflammatory-related pain, osteoarthritis (OA)-associated pain, chronic OA-related pain, and refractory OA-related pain.

30. The immunogenic vaccine of claim 29, wherein the pain is refractory to at least one anti-inflammatory compound selected from the group consisting of NSAID, a corticosteroid and an opioid analgesic.

31. The immunogenic vaccine of claim 27, wherein
(i) the pain is OA-associated pain,
(ii) the subject is a dog, and
(iii) the immunogenic recombinant fusion protein comprises
(a) an immunogenic recombinant fusion protein having SEQ ID NO: 27 and the vaccine elicits production of neutralizing antibodies against NGF and/or SP in the dog, or
(b) an immunogenic recombinant fusion protein having SEQ ID NO: 29 and the vaccine elicits production of neutralizing antibodies against NGF and/or CGRP in the dog.

32. (canceled)

33. The immunogenic vaccine of claim 31 formulated for subcutaneous administration followed by 3 booster injections administered approximately 2 weeks apart.
